(19) United States
(12) Patent Application Publication
(10) Pub. No.: US 2026/0345605 A1
(43) Pub. Date: Nov. 5, 2026

(54) THERAPEUTIC AGENTS FOR HEART FAILURE

(71) Applicant: Ottawa Hospital Research Institute, Ottawa (CA)

(72) Inventors: Lynn MEGENEY, Russell (CA); Anupam SHARMA, Ottawa (CA); William STANFORD, Ottawa (CA); Julien YOCKELL-LELIEVRE, Gatineau (CA)

(21) Appl. No.: 19/478,402

(22) PCT Filed: May 16, 2024
(86) PCT No.: PCT/CA2024/050658

§ 371 Date: Oct. 24, 2025

Related U.S. Application Data
(60) Provisional application No. 63/502,672, filed on May 17, 2023.

(51) Int. Cl. A61K 31/506; A61P 9/00; A61P 9/04

(52) U.S. Cl. CPC A61K 31/506; A61P 9/00; A61P 9/04

(57) ABSTRACT

Herein are described applications of AZD1152 (Barasertib) or LY2835219 (Abemaciclib) in relation to heart failure and/or stimulation of eccentric hypertrophy or sarcomere proliferation in series. AZD1152 or LY2835219 may be used for promoting therapeutic cardiac remodeling. Methods, uses, compositions, pharmaceutical compositions, kits, and commercial packages are provided.

### CROSS REFERENCE TO RELATED APPLICATIONS

This application claims the benefit of priority from U.S. Provisional Application No. 63/502,672 entitled “THERAPEUTIC AGENTS FOR HEART FAILURE”, which was filed one May 17, 2023, and which is incorporated by reference in its entirety.

### FIELD

The present disclosure relates generally to heart failure. More particularly, the present disclosure relates to agents relevant to treatment of heart failure.

### BACKGROUND

Heart failure is a major cause of death. Heart failure can be due to a variety of causes including congenital malformations, myocardial infarction, hypertension, ischemia, and dilated cardiomyopathies. Regardless of the variable causes of heart failure, there is a common pathologic progression that begins with a phase of maladaptive cardiac remodeling (for example, hypertrophy) whereby individual cardiac muscle cells increase in size as an attempted means of compensating for reduced cardiac pump function, but the etiology of this process is maladaptive and does not result in a stronger heart muscle.

Depending on the stimulus, cardiac hypertrophy can be a beneficial or non-beneficial adaptation. In a healthy population, cardiac remodeling is an adaptive measure that the heart takes in response to mechanical and molecular stimuli such as development, exercise, and pregnancy to maintain normal cardiac function. Also referred to as physiological hypertrophy, it typically entails a balanced increase in muscle mass and chamber volume of both ventricles with improved contractility, and serves to be beneficial in maintaining normal cardiac function. Physiological hypertrophy is also a reversible process, where the heart returns to baseline dimensions and function upon cessation of the growth challenge.

Pathological hypertrophy, on the other hand, is an irreversible maladaptive form of remodeling marked by an increase in heart size with no corresponding enhancement in function leading to detrimental effects on cardiac function, which eventually progresses towards heart failure.

Ventricular hypertrophy is associated with changes in myocyte morphology which has been classified into eccentric and concentric forms of hypertrophy based on the structural remodeling of cardiomyocytes.

Pathological stimuli (pressure overload, hypertension, aortic stenosis) generally result in the concentric form of hypertrophy where cardiomyocytes mostly increase in width compared to length because of the addition of sarcomeres in parallel, resulting in ventricular wall and septal thickening and a loss of chamber area. Eccentric hypertrophy, on the other hand, is induced by physiological stimuli (exercise, pregnancy) where the heart grows uniformly with cardiomyocyte sarcomere addition in series, which leads to increase in myocyte length compared to width.

Ventricular failure manifests in many forms, its underlying physiology ranging from overt left ventricular (LV) systolic dysfunction to isolated right ventricular (RV) diastolic dysfunction. Although RV and LV failure demonstrate important differences in etiology, they share many common characteristics when they adapt to adverse loading or when they fail. One of these features includes the morphometric changes in length and width of cardiomyocytes which eventually results in beneficial and non-beneficial adaption to stress stimuli.

The beneficial form of hypertrophy is generally observed in endurance athletes wherein the physiological remodeling resulted in high maximal oxygen uptake (VO2max) along with improved excitation-contraction coupling, larger blood volume that increases central venous pressure, increased myocardial endothelial function and vascularity, and improved metabolic capacity of the myocardium. Some reports also suggest that exercise training reverses pathological hypertrophy associated with heart failure and significantly improves cardiac function. Since this kind of preconditioning under a pathological setting confers physiological hypertrophic response, strategies involving reversion of detrimental hypertrophy towards beneficial hypertrophic response can be a potential therapeutic option in the management of heart failure. This approach is different from the current therapeutic strategies which are aimed at restricting hypertrophic growth, and is based on the idea that hypertrophic growth is non-essential for the initial short-term adaptation to pathological stimuli like pressure overload.

In contrast to physiologic hypertrophy, pathologic hypertrophy results in fetal gene re-expression, cardiomyocyte apoptosis and fibrosis and, after a period of compensatory function, progressive decline in left ventricular performance and, ultimately, heart failure.

Several small molecule cell cycle inhibitors have been reported to improve cardiac function, however their application in limiting pathological remodeling and favoring beneficial adaptation has not yet been tested. Recent studies identified CDK2 activation as a critical mechanism underlying DOX-induced cardiomyocyte apoptosis and ventricular dysfunction. Roscovitine, a small molecule CDK2 inhibitor was able to instigate cardioprotective mechanism by attenuating myocytes from DOX-associated contractile dysfunction and ventricular remodeling. Another study reported Palbociclib (CDK4/6 inhibitor) for inhibiting streptozotocin (STZ)-induced cardiac dysfunction by attenuating maladaptive cardiac remodeling in animal model of diabetic cardiomyopathy. However, the potential of these small molecule inhibitors in managing the maladaptive cardiac remodeling which is central to most of the cardiovascular diseases has not yet been tested.

There remains a need for therapeutics for heart failure.

### SUMMARY

It is an object of the present disclosure to obviate or mitigate at least one disadvantage of previously attempted treatments.

In one aspect, there is provided a method of promoting cardiac remodeling in a subject comprising administering to a subject a therapeutic agent comprising AZD1152-HQPA (Barasertib).

In one aspect, there is provided a method of promoting cardiac remodeling in a subject comprising administering to a subject a therapeutic agent comprising LY2835219 (Abemaciclib).

In one aspect, there is provided a method of treating heart failure in a subject comprising administering to a subject a therapeutic agent comprising AZD1152-HQPA (Barasertib).

In one aspect, there is provided a method of treating heart failure in a subject comprising administering to a subject a therapeutic agent comprising LY2835219 (Abemaciclib).

In one aspect, the present disclosure provides a method of stimulating eccentric hypertrophy of myocytes comprising contacting the myocytes with AZD1152-HQPA (Barasertib).

In one aspect, the present disclosure provides a method of stimulating eccentric hypertrophy of myocytes comprising contacting the myocytes with LY2835219 (Abemaciclib).

In one aspect, the present disclosure provides a method of stimulating sarcomere addition in series comprising contacting a sarcomere with AZD1152-HQPA (Barasertib). In one embodiment, the sarcomere is a cardiac sarcomere.

In one aspect, the present disclosure provides a method of stimulating sarcomere addition in series comprising contacting a sarcomere with LY2835219 (Abemaciclib). In one embodiment, the sarcomere is a cardiac sarcomere.

In one aspect, the present disclosure provides a use of AZD1152-HQPA (Barasertib) for inducement of cardiac remodeling in a subject.

In one aspect, the present disclosure provides a use of LY2835219 (Abemaciclib) for inducement of cardiac remodeling in a subject.

In one aspect, the present disclosure provides a use of AZD1152-HQPA (Barasertib) for treatment of heart failure in a subject.

In one aspect, the present disclosure provides a use of LY2835219 (Abemaciclib) for treatment of heart failure in a subject.

In one aspect the present disclosure provides a use of AZD1152-HQPA (Barasertib) for preparation of a medicament for inducement of cardiac remodeling in a subject.

In one aspect, the present disclosure provides a use of LY2835219 (Abemaciclib) for preparation of a medicament for inducement of cardiac remodeling in a subject.

In one aspect, the present disclosure provides a use of AZD1152-HQPA (Barasertib) for preparation of a medicament for treatment of heart failure in a subject.

In one aspect, the present disclosure provides a use of LY2835219 (Abemaciclib) for preparation of a medicament for treatment of heart failure in a subject.

In one aspect, the present disclosure provides a compound AZD1152-HQPA (Barasertib) for use in inducement of cardiac remodeling in a subject.

In one aspect, the present disclosure provides a compound LY2835219 (Abemaciclib) for use in inducement of cardiac remodeling in a subject.

In one aspect, the present disclosure provides a compound AZD1152-HQPA (Barasertib) for use in treatment of heart failure in a subject.

In one aspect, the present disclosure provides a compound LY2835219 (Abemaciclib) for use in treatment of heart failure in a subject.

In one aspect, the present disclosure provides a use of AZD1152-HQPA (Barasertib) for stimulating eccentric hypertrophy of myocytes.

In one aspect, the present disclosure provides AZD1152-HQPA (Barasertib) for use in stimulating eccentric hypertrophy of myocytes.

In one aspect, the present disclosure provides a use of LY2835219 (Abemaciclib) for stimulating eccentric hypertrophy of myocytes.

In one aspect, the present disclosure provides LY2835219 (Abemaciclib) for use in stimulating eccentric hypertrophy of myocytes.

In one aspect, the present disclosure provides a use of AZD1152-HQPA (Barasertib) for stimulating sarcomere addition in series.

In one aspect, the present disclosure provides AZD1152-HQPA (Barasertib) for use in stimulating sarcomere addition in series.

In one aspect, the present disclosure provides a use of LY2835219 (Abemaciclib) for stimulating sarcomere addition in series.

In one aspect, the present disclosure provides LY2835219 (Abemaciclib) for use in stimulating sarcomere addition in series.

In one aspect, the present disclosure provides a pharmaceutical composition comprising AZD1152-HQPA (Barasertib) for use in inducement of cardiac remodeling in a subject.

In one aspect, the present disclosure provides a pharmaceutical composition comprising LY2835219 (Abemaciclib) for use in inducement of cardiac remodeling in a subject.

In one aspect, the present disclosure provides a use of AZD1152-HQPA (Barasertib) for treatment of heart failure in a subject.

In one aspect, the present disclosure provides a use of LY2835219 (Abemaciclib) for treatment of heart failure in a subject.

In one aspect, the present disclosure provides a composition comprising AZD1152-HQPA (Barasertib) for use in stimulating eccentric hypertrophy of myocytes.

In one aspect, the present disclosure provides a composition comprising LY2835219 (Abemaciclib) for use in stimulating eccentric hypertrophy of myocytes.

In one aspect, the present disclosure provides a composition comprising use of AZD1152-HQPA (Barasertib) for use in stimulating sarcomere addition in series.

In one aspect, the present disclosure provides a composition comprising LY2835219 (Abemaciclib) for use in stimulating sarcomere addition in series.

In one aspect, the present disclosure provides a kit comprising AZD1152-HQPA (Barasertib) together with instructions for use in inducement of cardiac remodeling in a subject.

In one aspect, the present disclosure provides a kit comprising LY2835219 (Abemaciclib) together with instructions for use in inducement of cardiac remodeling in a subject.

In one aspect, the present disclosure provides a commercial package comprising AZD1152-HQPA (Barasertib) together with instructions for use in inducement of cardiac remodeling in a subject.

In one aspect, the present disclosure provides a commercial package comprising LY2835219 (Abemaciclib) together with instructions for use in inducement of cardiac remodeling in a subject.

In one aspect, the present disclosure provides a kit comprising AZD1152-HQPA (Barasertib) together with instructions for treatment of heart failure in a subject.

In one aspect, the present disclosure provides a kit comprising LY2835219 (Abemaciclib) together with instructions for treatment of heart failure in a subject.

In one aspect, the present disclosure provides a commercial package comprising AZD1152-HQPA (Barasertib) together with instructions for treatment of heart failure in a subject.

In one aspect, the present disclosure provides a commercial package comprising LY2835219 (Abemaciclib) together with instructions for treatment of heart failure in a subject.

In one aspect, the present disclosure provides a kit comprising AZD1152-HQPA (Barasertib) together with instructions for use in stimulating eccentric hypertrophy of myocytes.

In one aspect, the present disclosure provides a kit comprising LY2835219 (Abemaciclib) together with instructions for use in stimulating eccentric hypertrophy of myocytes.

In one aspect, the present disclosure provides a kit comprising AZD1152-HQPA (Barasertib) together with instructions for use in stimulating sarcomere addition in series.

In one aspect, the present disclosure provides a commercial package comprising LY2835219 (Abemaciclib) together with instructions for use in stimulating sarcomere addition in series.

In one aspect, the present disclosure provides a kit comprising AZD1152-HQPA (Barasertib) together with instructions for use in stimulating eccentric hypertrophy of myocytes.

In one aspect, the present disclosure provides a commercial package comprising LY2835219 (Abemaciclib) together with instructions for use in stimulating eccentric hypertrophy of myocytes.

In one aspect, the present disclosure provides a commercial package comprising AZD1152-HQPA (Barasertib) together with instructions for use in stimulating sarcomere addition in series.

In one aspect, the present disclosure provides a commercial package comprising LY2835219 (Abemaciclib) together with instructions for use in stimulating sarcomere addition in series.

Other aspects and features of the present disclosure will become apparent to those ordinarily skilled in the art upon review of the following description of specific embodiments in conjunction with the accompanying figures.

### BRIEF DESCRIPTION OF THE DRAWINGS

Embodiments of the present disclosure will now be described, by way of example only, with reference to the attached Figures.

FIG. 1 is a waterfall plot of primary screen data.

FIG. 2A to 2C are plots of enrichment scores among the primary screen data.

FIG. 3 is a morphometric analysis of cardiomyocytes stimulated with different treatments.

FIG. 4A is a morphometric analysis of iPSC cardiomyocytes stimulated with different treatments.

FIG. 4B show quantifications of mean cardio myocyte length of control and treated cardiomyocytes.

FIG. 4C show quantifications of mean cardio myocyte length of control and treated cardiomyocytes.

FIG. 5A show quantifications of ProNT-BNP levels of control and treated cardiomyocytes.

FIG. 5B show quantifications of ProNT-BNP levels of control and treated cardiomyocytes.

FIG. 6A is morphometric analysis of cardiomyocytes stimulated with different treatments.

FIG. 6B shows mean nuclear intensity across various treatments.

FIG. 7A is morphometric analysis of cardiomyocytes stimulated with different treatments.

FIG. 7B shows mean nuclear intensity across various treatments.

FIG. 8A is morphometric analysis of cardiomyocytes stimulated with different treatments.

FIG. 8B shows mean nuclear intensity across various treatments.

FIG. 9 shows a proposed mechanism of action of AZD1152-HQPA and LY2835219 in the cell cycle.

FIG. 10 is a is a morphometric analysis of cardiomyocytes stimulated with different treatments.

FIG. 11 shows quantifications of peak amplitude, time to peak and area under the curve for control and treated cardiomyocytes.

FIG. 12 shows a line graph of oxygen consumption rate of control and treated cardiomyocytes over time.

FIG. 13 shows quantifications of Heart Weight/Body Weight for rats treated with PE, LY2835219 control, PBS control and PE+LY2835219.

FIG. 14 shows images of hearts of rats treated with PE, LY2835219 control, PBS control and PE+LY2835219.

FIG. 15 shows quantifications of myocardial morphology of the left ventricles of hearts of rats treated with PE, LY2835219 control, PBS control and PE+LY2835219.

FIG. 16 shows quantifications of in vivo fractional shortening for rats treated with PE, LY2835219 control, PBS control and PE+LY2835219.

FIG. 17 shows quantifications of in vivo ejection fraction for rats treated with PE, LY2835219 control, PBS control and PE+LY2835219.

FIG. 18 shows quantifications of in vivo stroke volume for rats treated with PE, LY2835219 control, PBS control and PE+LY2835219.

FIG. 19 shows quantifications of in vivo cardiac output for rats treated with PE, LY2835219 control, PBS control and PE+LY2835219.

FIG. 20 shows histological analysis of cardiac fibrosis in rats treated with PE, LY2835219 control, PBS control and PE+LY2835219.

FIG. 21 shows quantifications of histological analysis of cardiac fibrosis in rats treated with PE, LY2835219 control, PBS control and PE+LY2835219 as shown in FIG. 20.

FIG. 22 shows histological analysis of cardiac angiogenesis in rats treated with PE, LY2835219 control, PBS control and PE+LY2835219.

FIG. 23 shows quantifications of histological analysis of cardiac angiogenesis in rats treated with PE, LY2835219 control, PBS control and PE+LY2835219 as shown in FIG. 22.

### DETAILED DESCRIPTION

Generally, the present disclosure describes applications of AZD1152 (Barasertib) or LY2835219 (Abemaciclib) related to heart failure and/or stimulation of eccentric hypertrophy or sarcomere addition in series. AZD1152 or LY2835219 may be used for promoting therapeutic cardiac remodeling.

### Therapeutic Methods for Cardiac Remodeling or Heart Failure

In a first aspect, the present disclosure provides a method of promoting cardiac remodeling in a subject comprising administering to a subject a therapeutic agent comprising AZD1152-HQPA (Barasertib).

AZD1152 is a dihydrogen phosphate prodrug of a pyrazoloquinazoline Aurora kinase inhibitor. AZD1152-hydroxyquinazoline pyrazol anilide (HQPA) is converted rapidly to the active AZD1152-HQPA in plasma. AZD1152-HQPA is a strong and selective inhibitor of Aurora B.

In one aspect, the present disclosure provides a method of promoting cardiac remodeling in a subject comprising administering to a subject a therapeutic agent comprising LY2835219 (Abemaciclib).

LY2835219 is a drug that inhibits Cyclin D1/CDK4 and CDK6 with low nanomolar potency, and inhibits Rb phosphorylation resulting in a G1 arrest and inhibition of addition.

Embodiments that are described below may apply to the aspect involving AZD1152 and/or LY2835219.

By “cardiac remodeling” is meant the molecular, cellular, and/or interstitial changes that are manifested clinically as changes in the size, shape and function of the heart.

Where “promoting”, “inducement”, “stimulating” and similar terms are used herein, it will be understood an increase as compared to a control subject or corresponding sample in which AZD1152 or LY2835219 is not administered or used.

In one embodiment, the cardiac remodeling comprises left ventricular cardiac remodeling. In one embodiment, the left ventricular cardiac remodeling comprises left ventricular eccentric hypertrophy.

In one embodiment, the cardiac remodeling comprises right ventricular cardiac remodeling. In one embodiment, the right ventricular cardiac remodeling comprises right ventricular eccentric hypertrophy.

In one embodiment, the cardiac remodeling comprises reduction of cardiac fibrosis and/or improvement of cardiac angiogenesis.

In one embodiment, the subject is a mammal. In one embodiment, the subject is a human.

In one aspect, the present disclosure provides a method of treating heart failure in a subject comprising administering to a subject a therapeutic agent comprising AZD1152-HQPA (Barasertib).

In one aspect, the present disclosure provides a method of treating heart failure in a subject comprising administering to a subject a therapeutic agent comprising LY2835219 (Abemaciclib).

Embodiments that are described below may apply to the aspect involving AZD1152 and/or LY2835219.

In one embodiment, the heart failure comprises left ventricular heart failure. In one embodiment, the left ventricular heart failure comprises left ventricular maladaptive hypertrophy. In one embodiment, the left ventricular heart failure comprises left ventricular concentric hypertrophy.

In one embodiment, the heart failure comprises right ventricular heart failure. In one embodiment, the right ventricular heart failure comprises right ventricular maladaptive hypertrophy. In one embodiment, the right ventricular heart failure comprises right ventricular concentric hypertrophy.

In one embodiment, the heart failure is caused by myocardial infarction.

In one embodiment, the heart failure is caused by pulmonary arterial hypertension.

In one embodiment, the heart failure is caused by cardiac fibrosis or impaired cardiac angiogenesis.

In one embodiment, the heart failure is caused by aortic stenosis.

In one embodiment, the subject is a mammal. In one embodiment, the subject is a human.

In one embodiment of methods described herein, the step of administering the LY2835219 (Abemaciclib) is a step of orally administering.

In one embodiment of methods described herein, the step of administering the AZD1152-HQPA (Barasertib) is a step of intravenously administering.

### Methods for Stimulating Eccentric Hypertrophy or Sarcomere Addition in Series

In one aspect, the present disclosure provides a method of stimulating eccentric hypertrophy of myocytes comprising contacting the myocytes with AZD1152-HQPA (Barasertib).

In one aspect, the present disclosure provides a method of stimulating eccentric hypertrophy of myocytes comprising contacting the myocytes with LY2835219 (Abemaciclib).

Embodiments that are described below may apply to the aspect involving AZD1152 and/or LY2835219.

In one embodiment, the myocytes are cardiomyocytes.

In one aspect, the present disclosure provides a method of stimulating sarcomere addition in series comprising contacting a sarcomere with AZD1152-HQPA (Barasertib). In one embodiment, the sarcomere is a cardiac sarcomere.

By “addition in series” is meant the addition of sarcomeres resulting in an increase in cardiomyocyte length to width ratio for increasing contractility and chamber volume to maintain cardiac output.

In one aspect, the present disclosure provides a method of stimulating sarcomere addition in series comprising contacting a sarcomere with LY2835219 (Abemaciclib). In one embodiment, the sarcomere is a cardiac sarcomere.

### Therapeutic Uses in Cardiac Remodeling or Heart Failure

In one aspect, the present disclosure provides a use of AZD1152-HQPA (Barasertib) for inducement of cardiac remodeling in a subject.

In one aspect, the present disclosure provides a use of LY2835219 (Abemaciclib) for inducement of cardiac remodeling in a subject.

Embodiments that are described below may apply to the aspect involving AZD1152 and/or LY2835219.

In one embodiment, the cardiac remodeling comprises left ventricular cardiac remodeling. In one embodiment, the left ventricular cardiac remodeling comprises left ventricular eccentric hypertrophy.

In one embodiment, the cardiac remodeling comprises right ventricular cardiac remodeling. In one embodiment, the right ventricular cardiac remodeling comprises right ventricular eccentric hypertrophy.

In one embodiment, the cardiac remodeling comprises reduction of cardiac fibrosis and/or improvement of cardiac angiogenesis.

In one aspect, the present disclosure provides a use of AZD1152-HQPA (Barasertib) for treatment of heart failure in a subject.

In one aspect, the present disclosure provides a use of LY2835219 (Abemaciclib) for treatment of heart failure in a subject.

Embodiments that are described below may apply to the aspect involving AZD1152 and/or LY2835219.

In one embodiment, the heart failure comprises left ventricular heart failure. In one embodiment, the left ventricular heart failure comprises left ventricular maladaptive hypertrophy. In one embodiment, the left ventricular heart failure comprises left ventricular concentric hypertrophy.

In one embodiment, the heart failure comprises right ventricular heart failure. In one embodiment, the right ventricular heart failure comprises right ventricular maladaptive hypertrophy. In one embodiment, the right ventricular heart failure comprises right ventricular concentric hypertrophy.

In one embodiment, the heart failure is caused by myocardial infarction.

In one embodiment, the heart failure is caused by pulmonary arterial hypertension.

In one embodiment, the heart failure is caused by cardiac fibrosis or impaired cardiac angiogenesis.

In one embodiment, the heart failure is caused by aortic stenosis.

In one embodiment, the subject is a mammal. In one embodiment, the subject is a human.

In one aspect the present disclosure provides a use of AZD1152-HQPA (Barasertib) for preparation of a medicament for inducement of cardiac remodeling in a subject.

In one aspect, the present disclosure provides a use of LY2835219 (Abemaciclib) for preparation of a medicament for inducement of cardiac remodeling in a subject.

Embodiments that are described below may apply to the aspect involving AZD1152 and/or LY2835219.

In one embodiment, the cardiac remodeling comprises left ventricular cardiac remodeling. In one embodiment, the left ventricular cardiac remodeling comprises left ventricular eccentric hypertrophy.

In one embodiment, the cardiac remodeling comprises right ventricular cardiac remodeling. In one embodiment, the right ventricular cardiac remodeling comprises right ventricular eccentric hypertrophy.

In one embodiment, the cardiac remodeling comprises reduction of cardiac fibrosis and/or improvement of cardiac angiogenesis.

In one embodiment, the subject is a mammal. In one embodiment, the subject is a human.

In one aspect, the present disclosure provides a use of AZD1152-HQPA (Barasertib) for preparation of a medicament for treatment of heart failure in a subject.

In one aspect, the present disclosure provides a use of LY2835219 (Abemaciclib) for preparation of a medicament for treatment of heart failure in a subject.

Embodiments that are described below may apply to the aspect involving AZD1152 and/or LY2835219.

In one embodiment, the heart failure comprises left ventricular heart failure. In one embodiment, the left ventricular heart failure comprises left ventricular maladaptive hypertrophy. In one embodiment, the left ventricular heart failure comprises left ventricular concentric hypertrophy.

In one embodiment, the heart failure comprises right ventricular heart failure. In one embodiment, the right ventricular heart failure comprises right ventricular maladaptive hypertrophy. In one embodiment, the right ventricular heart failure comprises right ventricular concentric hypertrophy.

In one embodiment, the heart failure is caused by myocardial infarction.

In one embodiment, the heart failure is caused by pulmonary arterial hypertension.

In one embodiment, the heart failure is caused by cardiac fibrosis or impaired cardiac angiogenesis.

In one embodiment, the heart failure is caused by aortic stenosis.

In one embodiment, the subject is a mammal. In one embodiment, the subject is a human.

In one aspect, the present disclosure provides a compound AZD1152-HQPA (Barasertib) for use in inducement of cardiac remodeling in a subject.

In one aspect, the present disclosure provides a compound LY2835219 (Abemaciclib) for use in inducement of cardiac remodeling in a subject.

In one embodiment, the cardiac remodeling comprises left ventricular cardiac remodeling. In one embodiment, the left ventricular cardiac remodeling comprises left ventricular eccentric hypertrophy.

In one embodiment, the cardiac remodeling comprises right ventricular cardiac remodeling. In one embodiment, the right ventricular cardiac remodeling comprises right ventricular eccentric hypertrophy.

In one embodiment, the cardiac remodeling comprises reduction of cardiac fibrosis and/or improvement of cardiac angiogenesis.

In one embodiment, the subject is a mammal. In one embodiment, the subject is a human.

In one aspect, the present disclosure provides a compound AZD1152-HQPA (Barasertib) for use in treatment of heart failure in a subject.

In one aspect, the present disclosure provides a compound LY2835219 (Abemaciclib) for use in treatment of heart failure in a subject.

Embodiments that are described below may apply to the aspect involving AZD1152 and/or LY2835219.

In one embodiment, the heart failure comprises left ventricular heart failure. In one embodiment, the left ventricular heart failure comprises left ventricular maladaptive hypertrophy. In one embodiment, the left ventricular heart failure comprises left ventricular concentric hypertrophy.

In one embodiment, the heart failure comprises right ventricular heart failure. In one embodiment, the right ventricular heart failure comprises right ventricular maladaptive hypertrophy. In one embodiment, the right ventricular heart failure comprises right ventricular concentric hypertrophy.

In one embodiment, the heart failure is caused by myocardial infarction.

In one embodiment, the heart failure is caused by pulmonary arterial hypertension.

In one embodiment, the heart failure is caused by cardiac fibrosis or impaired cardiac angiogenesis.

In one embodiment, the heart failure is caused by aortic stenosis.

In one embodiment, the subject is a mammal. In one embodiment, the subject is a human.

In one embodiment of uses described herein, the LY2835219 (Abemaciclib) is for oral administration. In one embodiment of uses described herein, the LY2835219 (Abemaciclib) is formulated for oral administration.

In one embodiment of uses described herein, the AZD1152-HQPA (Barasertib) is for intravenous administration. In one embodiment of uses described herein, the AZD1152-HQPA (Barasertib) is formulated for intravenous administration.

### Uses in Stimulation of Eccentric Hypertrophy or Sarcomere Addition in Series

In one aspect, the present disclosure provides a use of AZD1152-HQPA (Barasertib) for stimulating eccentric hypertrophy of myocytes.

In one aspect, the present disclosure provides AZD1152-HQPA (Barasertib) for use in stimulating eccentric hypertrophy of myocytes.

Embodiments that are described below may apply to the aspect involving AZD1152 and/or LY2835219.

In one embodiment, the myocytes are cardiomyocytes.

In one aspect, the present disclosure provides a use of LY2835219 (Abemaciclib) for stimulating eccentric hypertrophy of myocytes.

In one aspect, the present disclosure provides LY2835219 (Abemaciclib) for use in stimulating eccentric hypertrophy of myocytes.

Embodiments that are described below may apply to either one of the aspects involving AZD1152 or LY2835219.

In one embodiment, the myocytes are cardiomyocytes.

In one aspect, the present disclosure provides a use of AZD1152-HQPA (Barasertib) for stimulating sarcomere addition in series. In one embodiment, the sarcomere is a cardiac sarcomere.

In one aspect, the present disclosure provides AZD1152-HQPA (Barasertib) for use in stimulating sarcomere addition in series. In one embodiment, the sarcomere is a cardiac sarcomere.

In one aspect, the present disclosure provides a use of LY2835219 (Abemaciclib) for stimulating sarcomere addition in series. In one embodiment, the sarcomere is a cardiac sarcomere.

In one aspect, the present disclosure provides LY2835219 (Abemaciclib) for use in stimulating sarcomere addition in series. In one embodiment, the sarcomere is a cardiac sarcomere.

### Pharmaceutical Formulations for Use in Cardiac Remodeling or Heart Failure

In one aspect, the present disclosure provides a pharmaceutical composition comprising AZD1152-HQPA (Barasertib) for use in inducement of cardiac remodeling in a subject.

In one aspect, the present disclosure provides a pharmaceutical composition comprising LY2835219 (Abemaciclib) for use in inducement of cardiac remodeling in a subject.

Embodiments that are described below may apply to the aspect involving AZD1152 and/or LY2835219.

In embodiment of the above, the pharmaceutical composition further comprises a pharmaceutically acceptable excipient, diluent, or carrier.

In one embodiment, the cardiac remodeling comprises left ventricular cardiac remodeling. In one embodiment, the left ventricular cardiac remodeling comprises left ventricular eccentric hypertrophy.

In one embodiment, the cardiac remodeling comprises right ventricular cardiac remodeling. In one embodiment, the right ventricular cardiac remodeling comprises right ventricular eccentric hypertrophy.

In one embodiment, the cardiac remodeling comprises reduction of cardiac fibrosis and/or improvement of cardiac angiogenesis.

In one aspect, the present disclosure provides a use of AZD1152-HQPA (Barasertib) for treatment of heart failure in a subject.

In one aspect, the present disclosure provides a use of LY2835219 (Abemaciclib) for treatment of heart failure in a subject.

Embodiments that are described below may apply to the aspect involving AZD1152 and/or LY2835219.

In one embodiment, the heart failure comprises left ventricular heart failure. In one embodiment, the left ventricular heart failure comprises left ventricular maladaptive hypertrophy. In one embodiment, the left ventricular heart failure comprises left ventricular concentric hypertrophy.

In one embodiment, the heart failure comprises right ventricular heart failure. In one embodiment, the right ventricular heart failure comprises right ventricular maladaptive hypertrophy. In one embodiment, the right ventricular heart failure comprises right ventricular concentric hypertrophy.

In one embodiment, the heart failure is caused by myocardial infarction.

In one embodiment, the heart failure is caused by pulmonary arterial hypertension.

In one embodiment, the heart failure is caused by cardiac fibrosis or impaired cardiac angiogenesis.

In one embodiment, the heart failure is caused by aortic stenosis.

In one embodiment, the subject is a mammal. In one embodiment, the subject is a human.

It is understood that pharmaceutical compositions can be formulated depending upon the mode of delivery desired. However, it is contemplated that the pharmaceutical composition may be formulated, in some embodiments, with one or more pharmaceutically acceptable excipients, which can include, for example, solubilizing agent, a buffer or buffering agent, a salt, or a combination thereof. Other excipients that may be used in a pharmaceutical composition include complex sugars, oils or waxes, stabilizing agents, viscosity enhancing agents, and preservatives, emulsifying agents, coloring agents, release agents, coating agents, sweetening agents, lubricants, or a combination thereof.

In one embodiment of pharmaceutical formulations described herein, the pharmaceutical formulations comprising the LY2835219 (Abemaciclib) is for oral administration. In one embodiment of pharmaceutical formulations described herein, the pharmaceutical formulations comprising the LY2835219 (Abemaciclib) is formulated for oral administration.

In one embodiment of pharmaceutical formulations described herein, the pharmaceutical formulations comprising the AZD1152-HQPA (Barasertib) is for intravenous administration. In one embodiment of pharmaceutical formulations described herein, the pharmaceutical formulations comprising the AZD1152-HQPA (Barasertib) is formulated for intravenous administration.

### Compositions for Eccentric Hypertrophy or Sarcomere Addition in Series

In one aspect, the present disclosure provides a composition comprising AZD1152-HQPA (Barasertib) for use in stimulating eccentric hypertrophy of myocytes.

In one aspect, the present disclosure provides a composition comprising LY2835219 (Abemaciclib) for use in stimulating eccentric hypertrophy of myocytes.

Embodiments that are described below may apply to the aspect involving AZD1152 and/or LY2835219.

In one embodiment, the composition further comprises an acceptable excipient, diluent, or carrier.

In one embodiment, the myocytes are cardiomyocytes.

In one aspect, the present disclosure provides a composition comprising use of AZD1152-HQPA (Barasertib) for use in stimulating sarcomere addition in series. In one embodiment, the sarcomere is a cardiac sarcomere.

In one aspect, the present disclosure provides a composition comprising LY2835219 (Abemaciclib) for use in stimulating sarcomere addition in series. In one embodiment, the sarcomere is a cardiac sarcomere.

Embodiments that are described below may apply to the aspect involving AZD1152 and/or LY2835219.

In embodiment of the above, the composition further comprises an acceptable excipient, diluent, or carrier.

### Kits & Commercial Packages Cardiac Remodeling or Heart Failure

In one aspect, the present disclosure provides a kit comprising AZD1152-HQPA (Barasertib) together with instructions for use in inducement of cardiac remodeling in a subject.

In one aspect, the present disclosure provides a kit comprising LY2835219 (Abemaciclib) together with instructions for use in inducement of cardiac remodeling in a subject.

In one aspect, the present disclosure provides a commercial package comprising AZD1152-HQPA (Barasertib) together with instructions for use in inducement of cardiac remodeling in a subject.

In one aspect, the present disclosure provides a commercial package comprising LY2835219 (Abemaciclib) together with instructions for use in inducement of cardiac remodeling in a subject.

Embodiments that are described below may apply to the aspect involving AZD1152 and/or LY2835219.

In one embodiment, the pharmaceutical composition further comprises a pharmaceutically acceptable excipient, diluent, or carrier.

In one embodiment, the cardiac remodeling comprises left ventricular cardiac remodeling. In one embodiment, the left ventricular cardiac remodeling comprises left ventricular eccentric hypertrophy.

In one embodiment, the cardiac remodeling comprises right ventricular cardiac remodeling. In one embodiment, the right ventricular cardiac remodeling comprises right ventricular eccentric hypertrophy.

In one embodiment, the cardiac remodeling comprises reduction of cardiac fibrosis and/or improvement of cardiac angiogenesis.

In one aspect, the present disclosure provides a kit comprising AZD1152-HQPA (Barasertib) together with instructions for treatment of heart failure in a subject.

In one aspect, the present disclosure provides a kit comprising LY2835219 (Abemaciclib) together with instructions for treatment of heart failure in a subject.

In one aspect, the present disclosure provides a commercial package comprising AZD1152-HQPA (Barasertib) together with instructions for treatment of heart failure in a subject.

In one aspect, the present disclosure provides a commercial package comprising LY2835219 (Abemaciclib) together with instructions for treatment of heart failure in a subject.

Embodiments that are described below may apply to the aspect involving AZD1152 and/or LY2835219.

In one embodiment, the heart failure comprises left ventricular heart failure. In one embodiment, the left ventricular heart failure comprises left ventricular maladaptive hypertrophy. In one embodiment, the left ventricular heart failure comprises left ventricular concentric hypertrophy.

In one embodiment, the heart failure comprises right ventricular heart failure. In one embodiment, the right ventricular heart failure comprises right ventricular maladaptive hypertrophy. In one embodiment, the right ventricular heart failure comprises right ventricular concentric hypertrophy.

In one embodiment, the heart failure is caused by myocardial infarction.

In one embodiment, the heart failure is caused by pulmonary arterial hypertension.

In one embodiment, the heart failure is caused by cardiac fibrosis or impaired cardiac angiogenesis.

In one embodiment, the heart failure is caused by aortic stenosis.

In one embodiment, the subject is a mammal. In one embodiment, the subject is a human.

Individual components of the kit or commercial package may be packaged in separate containers and, associated with such containers, can be a notice in the form prescribed by a governmental agency regulating the manufacture, use or sale of pharmaceuticals or biological products, which notice reflects approval by the agency of manufacture, use or sale for human administration.

In relation to any of the kits or commercial packages described herein, when the components of the kit or commercial package are provided in one or more liquid solutions, the liquid solution can be an aqueous solution, for example a sterile aqueous solution. In this case the container means may itself be an inhalant, syringe, pipette, eye dropper, or other such like apparatus, from which the composition may be administered to a patient.

The components of the kit or commercial package may also be provided in dried or lyophilised form and the lit can additionally contain a suitable solvent for reconstitution of the lyophilised components. Irrespective of the number or type of containers, the kits of the disclosure also may comprise an instrument for assisting with the administration of the composition to a patient. Such an instrument may be an inhalant, syringe, pipette, forceps, measured spoon, eye dropper or any such medically approved delivery vehicle.

The dosage form and, optionally, other components of the kit or commercial package, may be packaged in an appropriate container and, associated with such containers, can be a notice in the form prescribed by a governmental agency regulating the manufacture, use or sale of pharmaceutical or biological products, which notice reflects approval by the agency of manufacture, use or sale for human or animal administration. For example, LY2835219 (Abemaciclib) may be administered orally, while AZD1152-HQPA (Barasertib) may be administer orally or intravenously.

When the components of the kit or commercial package may be provided in one or more liquid solutions, the liquid solution can be an aqueous solution or suspension, for example, a sterile aqueous solution or suspension. In this case the container means may itself be an inhaler, syringe, pipette, eye dropper, nasal dropper, ear dropper, or other such like apparatus, from which the formulation may be administered to a patient. The container may also be a dry powder inhaler.

Irrespective of the number or type of containers, the kit or commercial package may comprise an instrument for assisting with the administration of the composition to a patient. Such an instrument may be an inhalant, syringe, pipette, forceps, measured spoon, eye dropper or any such medically approved delivery vehicle.

In one embodiment of kits and/or commercial packages described herein, the LY2835219 (Abemaciclib) is for oral administration. In one embodiment of kits and/or commercial packages described herein, the LY2835219 (Abemaciclib) is formulated for oral administration.

In one embodiment of kits and/or commercial packages described herein, the AZD1152-HQPA (Barasertib) is for intravenous administration. In one embodiment of kits and/or commercial packages described herein, the AZD1152-HQPA (Barasertib) is formulated for intravenous administration.

### Kits or Commercial Packages for Eccentric Hypertrophy or Sarcomere Addition in Series

In one aspect, the present disclosure provides a kit comprising AZD1152-HQPA (Barasertib) together with instructions for use in stimulating eccentric hypertrophy of myocytes. In one embodiment, the myocytes are cardiomyocytes.

In one aspect, the present disclosure provides a kit comprising LY2835219 (Abemaciclib) together with instructions for use in stimulating eccentric hypertrophy of myocytes. In one embodiment, the myocytes are cardiomyocytes.

In one aspect, the present disclosure provides a kit comprising AZD1152-HQPA (Barasertib) together with instructions for use in stimulating sarcomere addition in series. In one embodiment, the sarcomere is a cardiac sarcomere.

In one aspect, the present disclosure provides a commercial package comprising AZD1152-HQPA (Barasertib) together with instructions for use in stimulating sarcomere addition in series. In one embodiment, the sarcomere is a cardiac sarcomere.

In one aspect, the present disclosure provides a kit comprising LY2835219 (Abemaciclib) together with instructions for use in stimulating eccentric hypertrophy of myocytes. In one embodiment, the myocytes are cardiomyocytes.

In one aspect, the present disclosure provides a commercial package comprising LY2835219 (Abemaciclib) together with instructions for use in stimulating eccentric hypertrophy of myocytes. In one embodiment, the myocytes are cardiomyocytes.

In one aspect, the present disclosure provides a commercial package comprising AZD1152-HQPA (Barasertib) together with instructions for use in stimulating sarcomere addition in series. In one embodiment, the sarcomere is a cardiac sarcomere.

In one aspect, the present disclosure provides a commercial package comprising LY2835219 (Abemaciclib) together with instructions for use in stimulating sarcomere addition in series. In one embodiment, the sarcomere is a cardiac sarcomere.

### EXAMPLES

Experimental observations demonstrate that cardiomyocytes treated with AZD1152-HQPA (Barasertib) or LY2835219 (Abemaciclib) engage in a form of adaptive and compensatory remodeling that resembles a physiologic growth response, adding sarcomeres in series.

A library of 800 small molecule compounds that can stimulate favorable cardiac adaptation in primary neonatal rat ventricular myocytes (NRVM) was screened. To identify the beneficial modifiers of hypertrophy, the Cellomics Arrayscan High Throughput Screening platform was employed to quantify morphometric features such as, for example, length, width, and length to width ratio from images acquired using a 10× lens for DAPI-labeled nuclei and CY3 labeled α actinin stained cardiomyocytes in a 96 well format. The lead compound was also screened in iPSC derived cardiomyocytes as a part of secondary screening as it offers a physiologically relevant human cell-based model.

### Example 1: Screening Compounds for Hypertrophic Effect on Neonatal Rat Ventricular Myocytes

Neonatal rat ventricular myocytes (NRVM) were isolated from 2-day-old Sprague Dawley rat pups. The NRVM were cultured in plating media (Dulbecco Modified Eagle Media, 10% Fetal Bovine Serum, 100 U/mL penicillin, and 50 mg/mL streptomycin) at a density of 10,000 cells per well in a 96 well plate format. One day after isolation, myocytes were switched to serum-free maintenance media for 24 hours.

Eight hundred compounds from the Ontario Institute for Cancer Research (OICR library) and tool compound library were tested for their ability to induce physiological hypertrophy. All compounds were screened at concentrations 1 μM in duplicates, resulting in a total of 2000 wells in a 96 well plate format, including controls. Post-24 hour treatment, the cells were washed, fixed and stained with a Actinin for cardiomyocytes followed by nuclear staining with DAPI.

An algorithm was developed in Cellomics Arrayscan Vti HCS reader to measure the morphological characteristics such as cardiomyocyte length, width, length-width ratio, cell area, and the intensity of α-actinin. It was observed that treatment with hCT1 induced an increase in the length of cardiomyocyte when compared to PE-treated cardiomyocyte and a control. All the experiments were repeated 3 times.

Cardiomyocyte length was chosen as the primary parameter to identify compounds inducing the eccentric cardiac remodeling. The compounds were assigned a score (Z Score) and were ranked from high to low based on their ability to induce cardiomyocyte length. The compounds with Z scores comparable to control hCT1 treatments were selected for further investigation.

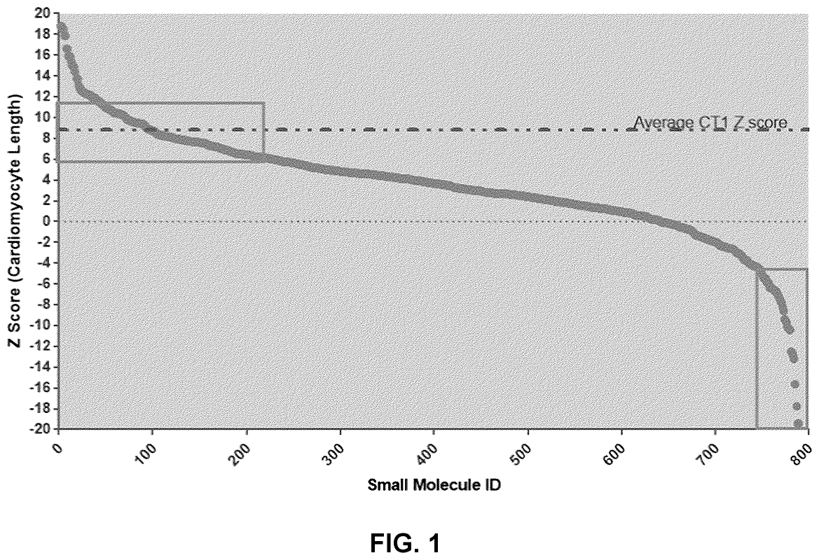

FIG. 1 shows a waterfall plot of primary screen data in which 800 compounds were tested in duplicate at a concentration of 1 μM for a total of 1600 data points. Results were normalized to Z-scores based on DMSO-treated wells. Cardiotrophin 1 at 40 ng/mL was used as a positive control. Compound with Z-score more than 10 were selected for the secondary screening.

Primary screening identified several cardiomyocyte length-promoting compounds from kinase inhibitor and tool compound library as shown in FIG. 1. Cell cycle inhibitor compounds were most prevalent in the screen.

### Example 2: Enrichment Profile Analysis

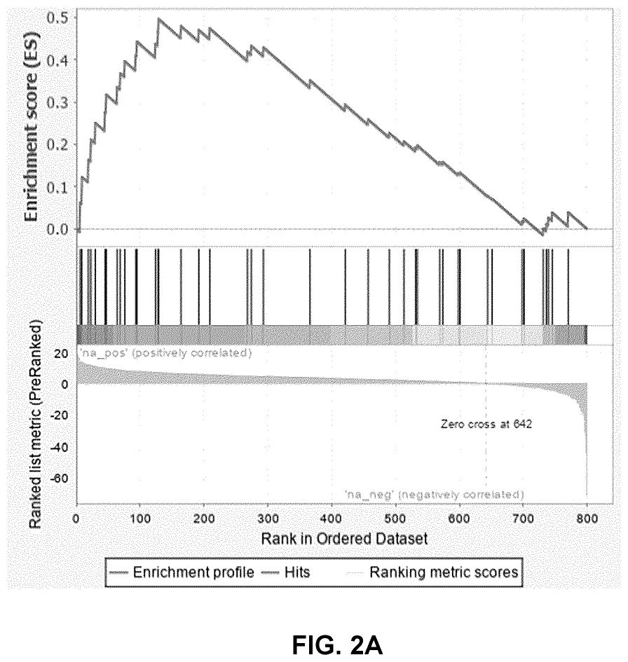

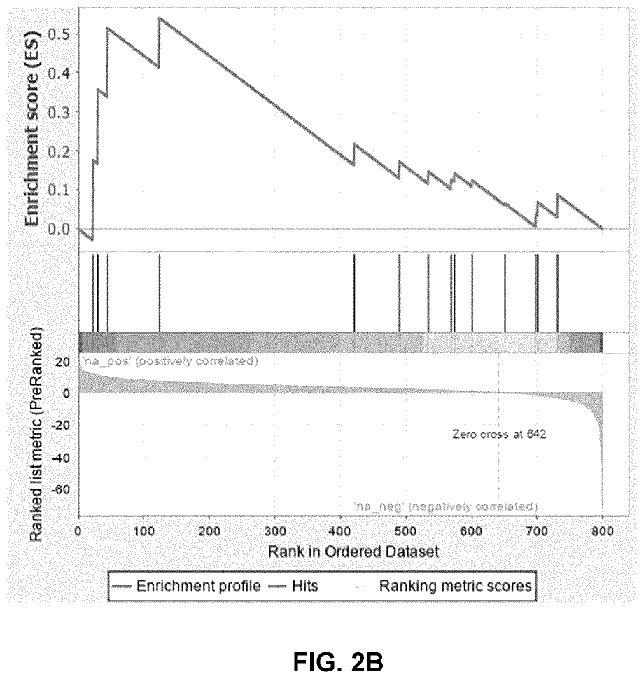

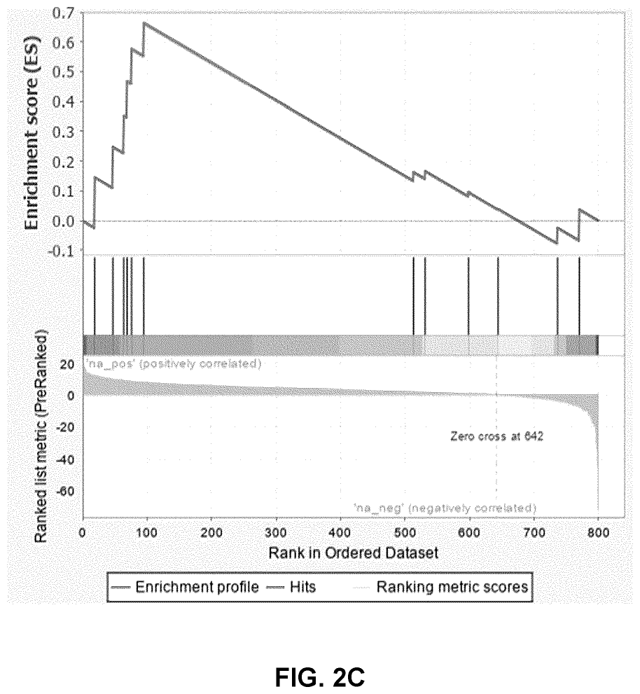

Enrichment of targets identified among the primary screen data was evaluated using known annotated targets of the employed compounds shown in FIG. 2A, 2B, 2C and Table 1. Target enrichment analysis was performed by adapting the Gene Set Enrichment Analysis (GSEA) algorithm using the GSEA software version 4.0.3. Distribution of length promoting activity of the compounds was evaluated within individual target classes to refine a small molecule list for further investigation. A weighted enrichment statistical analysis was carried out and the number of random permutations was set to 1,000 times. Inhibition of cell cycle pathway was identified to favour increase in the length of cardiomyocytes. The analysis further identified Aurora Kinase B and CDK4 to be the highly enriched molecular targets in the screen.

TABLE 1
Enrichment results via adaptation of gene set enrichment analysis (GSEA)
Gene set enrichment analysis

| | Drug | Enrichment | | False Discovery |
|---|---|---|---|---|
| Molecular Target | set size | score (ES) | p-value | Rate (FDR) |
| Cell Cycle | 41 | 0.495 | 0.001 | 0.054 |
| CDK4-Cyclin D1 | 12 | 0.663 | 0.007 | 0.101 |
| Aurora Kinase B | 14 | 0.54 | 0.059 | 0.343 |

### Example 3: Validation of Screen Results in NRVM

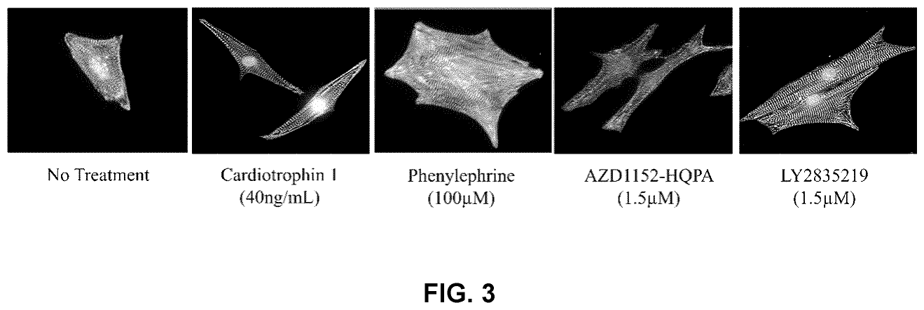

Observations were validated NRVM treated with AZD1152-HQPA and LY2835219 shown in FIG. 3. The effect of a control group and the drug were further validated using immunofluorescence. Primary NRVMs were seeded at a density of 2.7×105 cells directly onto 25 mm diameter collagen-coated glass coverslips. The treatments with controls (hCT1, PE) and drugs (AZD1152-HQPA, LY283519) were performed for 24 hours.

Post treatment, the cells were washed three times in PBS followed by fixation in 4% paraformaldehyde for 30 min. Cells were then permeabilized in blocking buffer (1% bovine serum albumin (BSA), 2% normal goat serum, 0.4% Tx-100 in PBS) for 15 min. Cells were then incubated for 2 h at room temperature with primary a Actinin antibody followed by three successive washes in PBS. Cells were then blocked again in blocking buffer for 15 min followed by a 1 hour incubation in fluorescently conjugated secondary antibody. Cells were then washed again in PBS and the nuclei were counterstained with 4′,6-diamidino-2-phenylindole (DAPI). The cells were mounted onto microscope slides with DakoCytomation fluorescent mounting medium (Dako). Digital fluorescent images were obtained using the Zeiss AxioVision SE64 Imaging software and analyzed using the ImageJ2 software.

### Example 4: Secondary Screening

A secondary screening was performed with 10 drugs identified from primary screening for their ability to induce physiological remodeling using morphometric analysis and measuring the levels of B-type natriuretic peptide (BNP), as a surrogate indicator of the hypertrophic response using ELISA. The compounds were selected based on the highly enriched targets identified in the drug set enrichment analysis with a high Z score. Cryopreserved iPS cell-derived cardiomyocytes (“iCell Cardiomyocytes”) obtained from Cellular Dynamics International (CDI, Madison, WI) were used as a cell model to perform the screening.

The cells were plated at a density of 2.5×10⁴ cells onto 25 mm diameter fibronectin coated glass coverslips in plating media for 48 hours. The media was replaced with Supplemented William's E (SWE) medium (SWE) media supplemented with cocktail containing penicillin-streptomycin, ITS+(insulin, transferrin, selenium complex, BSA, and linoleic acid), GlutaMAX™, and HEPES (Primary Hepatocyte Maintenance Supplements-GIBCO) for 48 hours. The cells were treated with controls (CT1-40 ng/mL, PE-100 uM) and drugs (AZD1152-HQPA, LY283519) at varying concentrations (2.5, 1, 0.5, and 0.25 uM) for 24 hours.

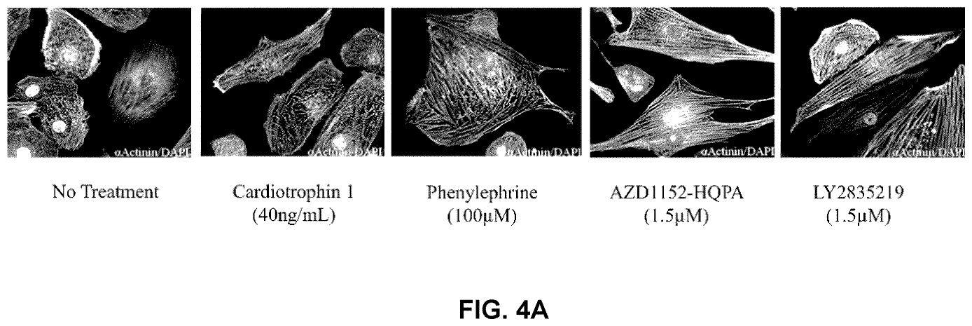

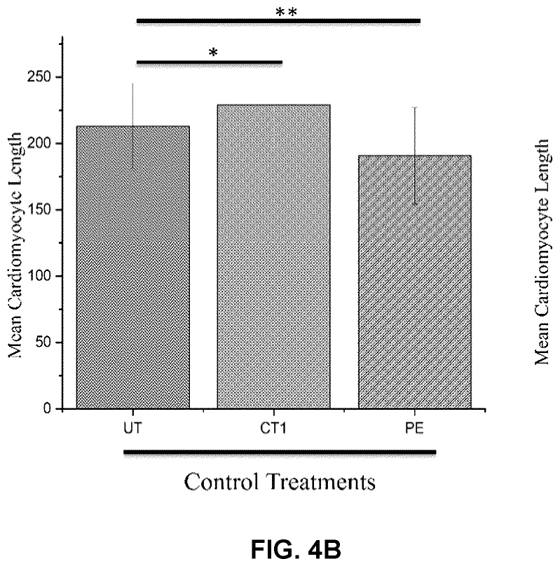

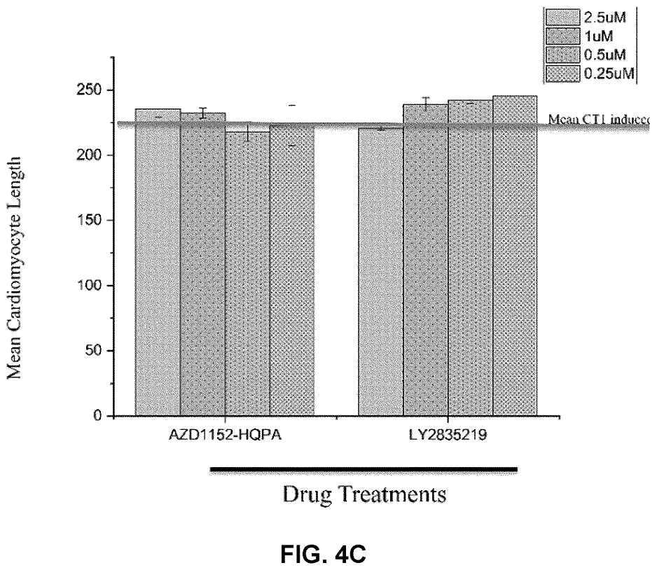

Post treatments, the cells were washed, fixed and treated with primary antibody, secondary antibody similar to the protocol of Example 3. The effect on the morphometric parameters was assessed using Cellomics Arrayscan Vti HCS reader. The Image acquisition was performed using the Zeiss AxioVision SE64 Imaging software and analyzed using the ImageJ2 software. AZD1152.HQPA and LY2835219 treated cells had comparable cardiomyocyte length with CT1 treated cells. Eccentric cardiac remodeling was observed in iCell cardiomyocyte following treatment with both AZD1152-HQPA (1 uM) and LY2835219 (1 uM) as shown in FIG. 4A.

iCell cardiomyocytes were seeded at a density of 10,000 cells per well in a 96 well plate format. The cells were maintained as in Example 3 and were treated with controls (hCT1, PE) and drugs (AZD1152-HQPA, LY283519) at four different concentrations for 24 hours. The cells were then fixed and stained with a Actinin for cardiomyocytes followed by nuclear staining with DAPI. The plates were analyzed in Cellomics Arrayscan Vti HCS reader to assess morphometric changes following treatment. The mean cardiomyocyte length induced by the drugs were compared to the mean cardiomyocyte length of hCT1 treated cardiomyocyte which is a positive modulator of cardiomyocyte length.

AZD1152-HQPA and LY2385219 promoted increased length in iCell cardiomyocyte comparable to length induced by hCT1 as shown in FIG. 4A, FIG. 4B, and Table 2.

TABLE 2
Secondary validation of drugs to promote
cardiomyocyte length in iCell Cardiomyocyte.
Mean Cardiomyocyte Length

| | Mean Cardiomyocyte | Standard Error |
|---|---|---|
| Treatments | Length (μM) | of Mean (SEM) |
| UT | 213.01323 | 8.33 |
| CT1 | 228.90683 | 8.15 |
| PE | 190.66018 | 9.41 |
| AZD1152-HQPA (0.25 μm) | 235.83305 | 3.94 |
| AZD1152-HQPA (0.5 μm) | 232.32049 | 2.22 |
| AZD1152-HQPA (1 μm) | 218.0957 | 4.43 |
| AZD1152-HQPA (2.5 μm) | 222.68344 | 8.92 |
| LY2835219 (0.25 μm) | 220.73547 | 0.86 |
| LY2835219 (0.5 μm) | 238.74493 | 3.16 |
| LY2835219 (1 μm) | 242.43285 | 1.67 |
| LY2835219 (2.5 μm) | 245.41377 | 1.73 |

### Example 5: B-Type Natriuretic Peptide Measurement

B-type natriuretic peptides (BNP or NT-proBNP) have been studied extensively in congestive heart failure patients, and their expression is an accepted indicator of the hypertrophic response in cardiomyocytes.

Pro-NT BNP levels in the culture media were measured following the 24 hour treatment using the Human NT Pro-BNP DuoSet ELISA kit (R&D Systems, Minnesota USA) as per the manufacturer's instruction. On day 5 post-thaw and following treatments with controls (hCT1, PE) and drug (AZD1152-HQPA, LY2835219) for 24 hours, supernatants from the cardiomyocyte cell culture plate were analyzed by a sandwich ELISA for the detection of secreted BNP using the Human NT Pro-BNP DuoSet ELISA kit (R&D Systems, USA) as per the manufacturers instruction. The plates were read on BMG BioTek Molecular Devices SpectraMax Microplate Reader at 450 nm to obtain the optical density followed by reading at 540 nm for wavelength correction.

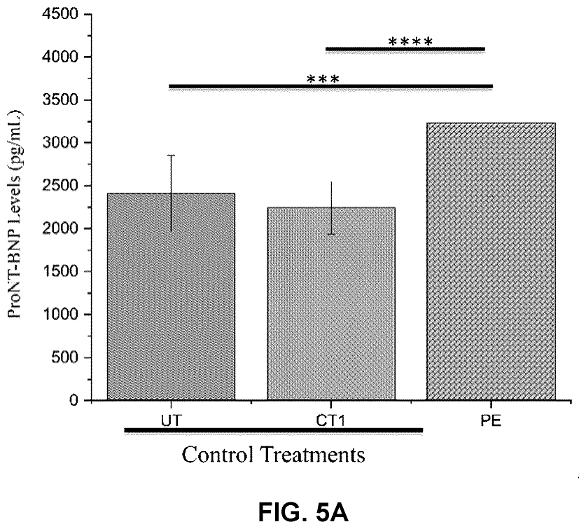

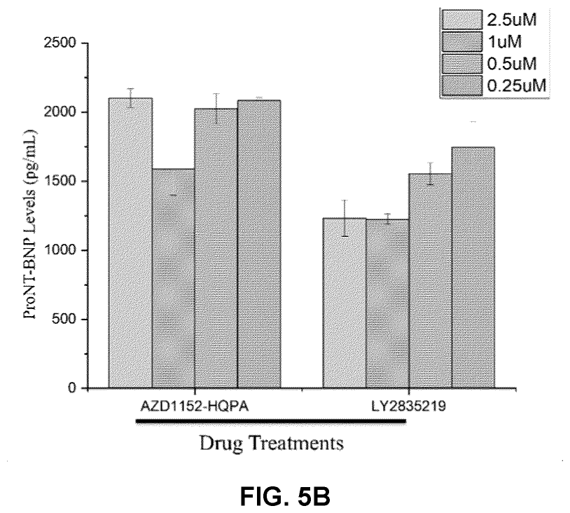

Protein levels were identified by plotting a 4 parametric logistic regression curve using the standards provided with the kit. Pro-NT BNP levels in control groups showed were significantly elevated levels in phenylephrine (PE) treated group compared to both UT and CT1 treated cells. Pro-NT BNP levels in drug-treated groups identified AZD1152-HQPA and LY2835219 to have comparable and/or reduced BNP levels as compared to the UT and CT1 treated groups as shown in as shown in FIG. 5A, FIG. 5B, and Table 3.

TABLE 3
Mean NT Pro-BNP levels in iCell
Cardiomyocyte following drug treatment.
Mean NT Pro-BNP levels (ELISA)

| | Mean NT Pro-BNP | Standard Error |
|---|---|---|
| Treatments | levels (pg/mL) | of Mean (SEM) |
| UT | 2407.521 | 111.69 |
| CT1 | 2240.774 | 76.91 |
| PE | 3236.015 | 217.75 |
| AZD1152-HQPA (0.25 μm) | 2100.945 | 39.41 |
| AZD1152-HQPA (0.5 μm) | 1588.366 | 109.57 |
| AZD1152-HQPA (1 μm) | 2023.151 | 111.02 |
| AZD1152-HQPA (2.5 μm) | 2085.823 | 12.84 |
| LY2835219 (0.25 μm) | 1232.484 | 76.32 |
| LY2835219 (0.5 μm) | 1226.226 | 22.71 |
| LY2835219 (1 μm) | 1554.354 | 46.94 |
| LY2835219 (2.5 μm) | 1747.475 | 106.2 |

### Example 6: AZD1152-HQPA and Cardiac Remodeling

Immunofluorescence was performed to validate and assess the effect of aurora B kinase inhibitor on cardiomyocyte remodeling. NRVMs were plated at a density of 2.5×10⁴ cells onto 25 mm diameter collagen coated coverslip. Primary cardiomyocytes were incubated with control serum-free medium (Ctrl, SF medium), hCT1 (0.5 nM), PE (100 μM), AZD1152-HQPA (1.5 μM) and assessed for aurora kinase B activation using pAurkB s727 antibody staining. The cells were treated with a combination of PE and AZD1152 HQPA where the cells were stimulated with the drug for 4 hours following the PE treatment for 24 hours.

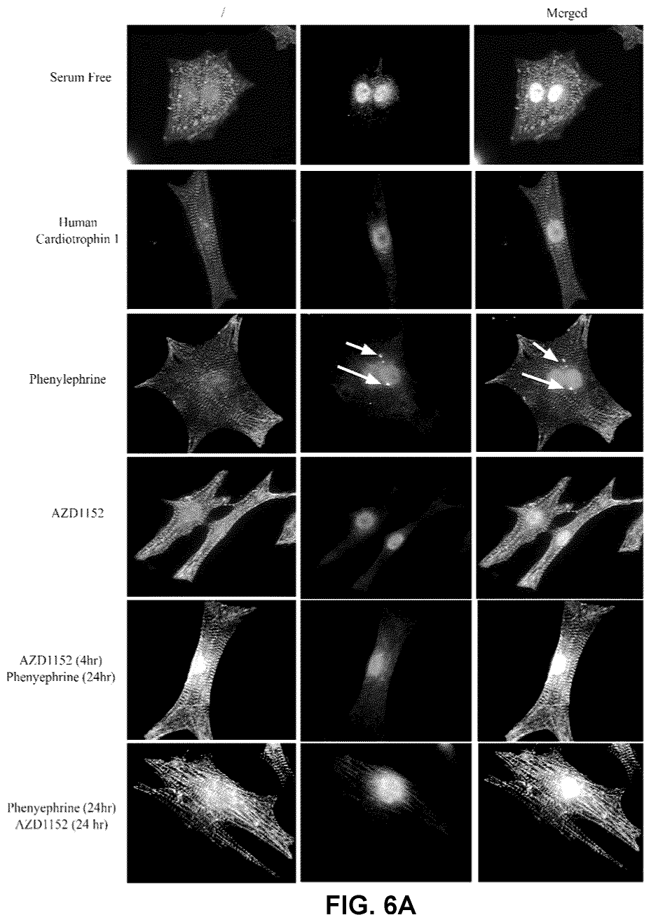

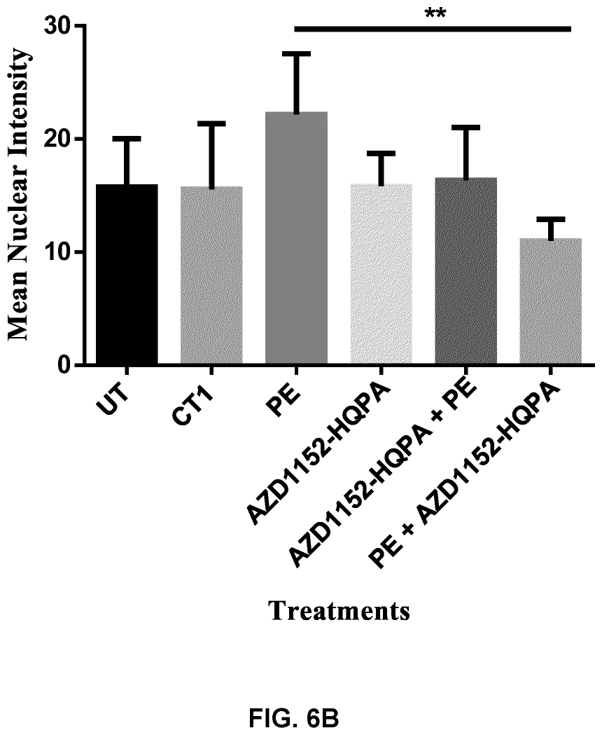

Post treatment, the cells were washed, fixed, and were subjected to sequential staining of primary antibodies a Actinin and phospho-AurkB (S727). The images were acquired using the Zeiss AxioVision SE64 Imaging software and analyzed using the ImageJ2 software. Immunofluorescence was used to detect α-actinin, pAurkb S727, and nuclei were stained with DAPI. AurkB activation along with concentric hypertrophic remodeling was observed in PE treated cells indicating the kinase activation in response to pathological stimuli. AZD1152-HQPA inhibited aurora kinase b activation was able to reverse the pathological effect of PE As seen in FIG. 6A and FIG. 6B.

### Example 7: Statistical Significance Assessment

A one-way ANOVA test performed to assess statistical significance across treatment groups.

The effect of AZD1152-HQPA on cardiac remodeling was further validated using the phospho-Histone 3 (pH3 Ser10) staining which is the downstream target of aurora kinase B. The NRVMs were plated at a density of 2.5×104 cells onto 25 mm diameter collagen coated coverslip. The cells were treated with controls (hCT1, PE), AZD1152-HQPA followed by a combination of PE and AZD1152-HQPA.

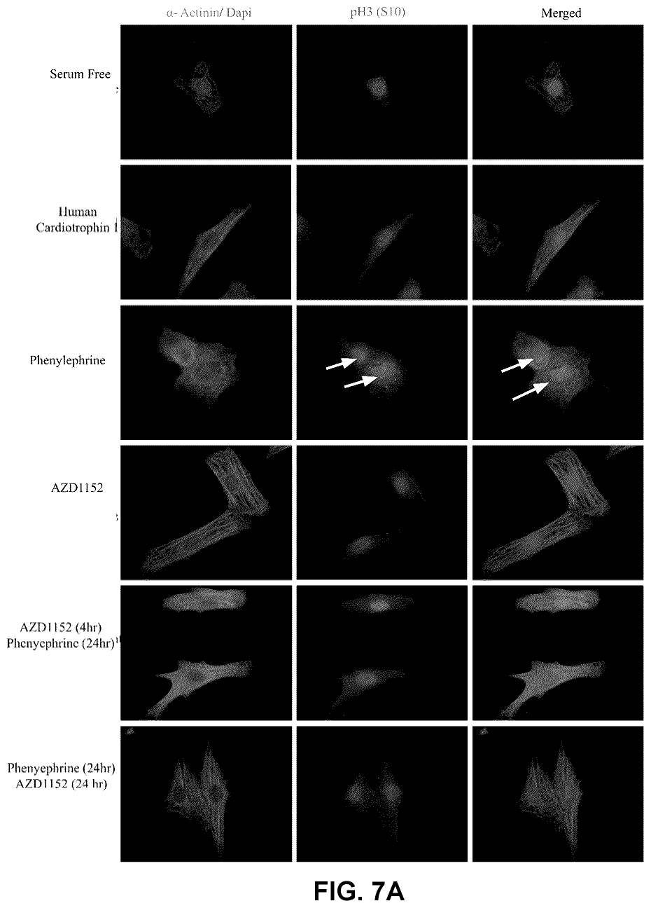

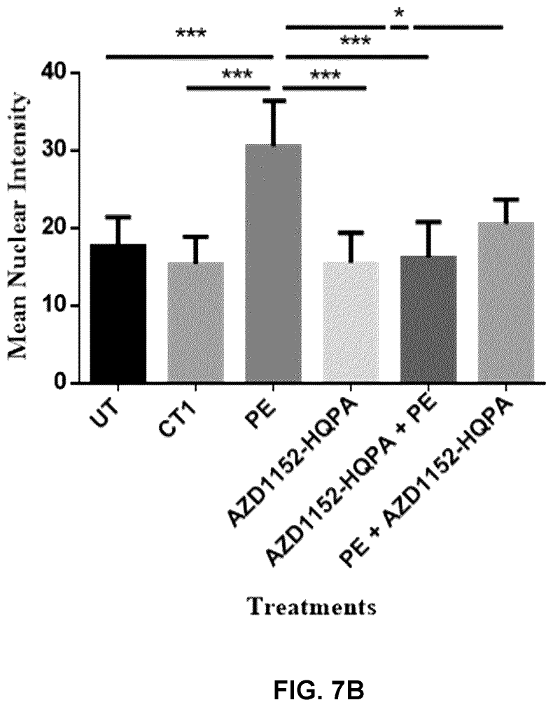

Post treatment, the cells were washed, fixed, and were subjected to sequential staining of primary antibodies a Actinin and phospho-Histone 3 (Ser10). The images were acquired using the Zeiss AxioVision SE64 Imaging software and analyzed using the ImageJ software. Increased pH3 Ser10 staining was observed in NRVM treated with PE while AZD1152-HQPA was marked with reduced pH3 activity both in the treatment group alone and in combination with PE treatment (FIG. 6). The data are represented as Mean±Standard deviation. One-way ANOVA tests were performed to assess statistical significance across treatment groups as seen in FIGS. 7A and 7B.

### Example 8: LY2835219 and Cellular Cardiac Remodeling

Primary cardiomyocytes were incubated with control serum-free medium (Ctrl, SF medium), hCT1 (0.5 nM), PE (100 μM), LY2835219 (1.5 μM) and assessed for CDK4 activity using pCDK4 Thr172 antibody staining. The cells were also treated with a combination of PE and LY2835219 where the cells were stimulated with the drug for 4 hr following the PE treatment for 24 hours. Post treatment, the cells were washed, fixed, and were subjected to sequential staining of primary antibodies a Actinin and phospho-CDK4 (Thr172). The images were acquired using the Zeiss AxioVision SE64 Imaging software and analyzed using the ImageJ2 software.

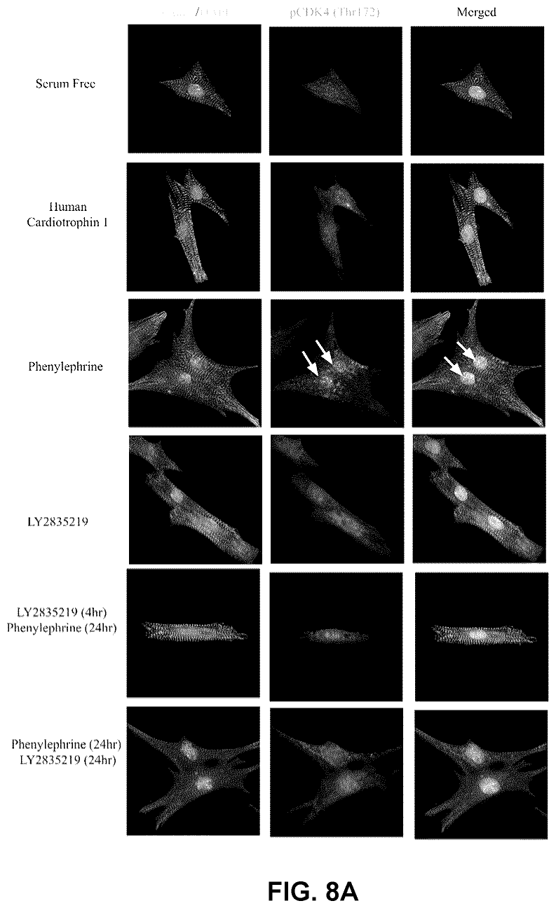

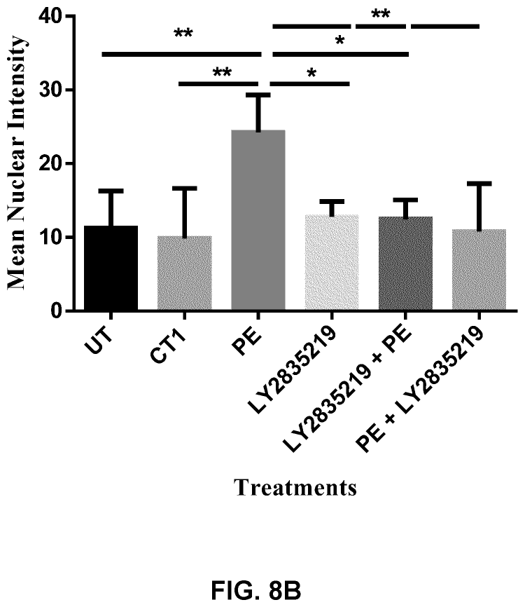

Immunofluorescence was used to detect α-actinin, pCDK4 Thr172, and nuclei were stained with DAPI. CDK4 activation along with concentric hypertrophic remodeling was observed in PE treated cells indicating the kinase activation in response to pathological stimuli. Ly2835219 inhibited CDK4 activation and was able to reverse the pathological effect of PE (FIG. 7). The nuclear fluorescent intensity of pCDK4 Thr172 using the ImageJ software. The data are represented as Mean±Standard deviation. One-way ANOVA tests were performed to assess statistical significance across treatment groups as shown in FIG. 8A and FIG. 8B.

### Mechanism of Action

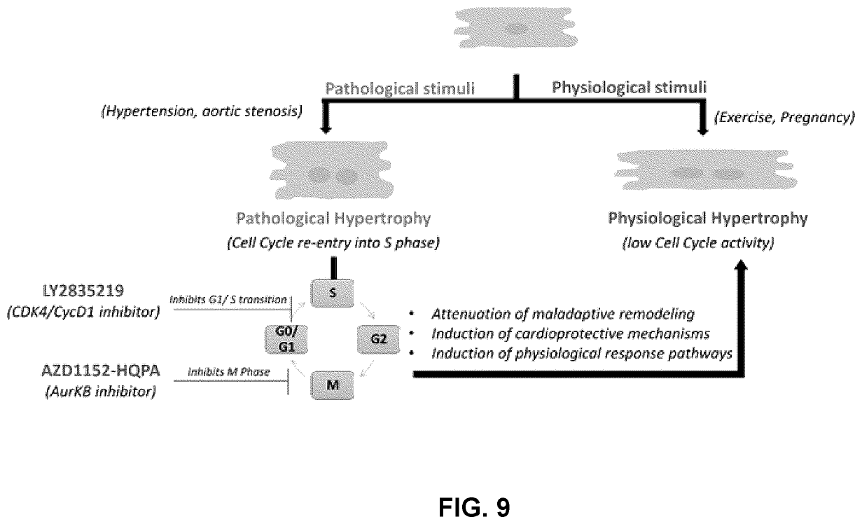

FIG. 9 shows the proposed mechanism of action of drugs AZD1152-HQPA and LY2835219. Pathological stimuli may induce a maladaptive form of cardiomyocyte remodeling followed by re-entry to the cell cycle which may progress towards heart failure. Cell cycle re-entry which is reported as a catastrophic event may be inhibited by AZD1152-HQPA and LY2835218 at the M phase and G1/S phase of cell cycle to restore cardiac function. These drugs may not only inhibit the pathological form of cardiac remodeling but may also transition the pathological features towards physiological phenotype by inducing physiological response pathways

### Example 9: LY2835219 and Cardiac Extracellular Matrix (ECM) Remodeling

The extracellular matrix (ECM) is an essential component of the heart that imparts fundamental cellular processes during organ development and homeostasis. Pathological cardiac hypertrophy involves severe remodeling of the ECM, culminating in the formation of fibrotic tissue that is deleterious to organ function. Fibrotic events include increased expression of collagen type I, which leads to mechanical stiffness, eventually leading to cardiac dysfunction.

Primary NRVMs were seeded at a density of 2.7×105 cells directly onto 25 mm diameter collagen-coated glass coverslips. The treatments with control (PE) and drugs LY283519 were performed for 24 hours. For the combination treatments, the cells were treated with PE for 24 hrs followed by drug LY2835219 for 24 hrs. Post treatment, the cells were washed three times in PBS followed by fixation in 4% paraformaldehyde for 30 min. Cells were then permeabilized in blocking buffer (1% bovine serum albumin (BSA), 2% normal goat serum, 0.4% Tx-100 in PBS) for 15 min and were incubated for 2 h at room temperature with primary a Actinin and collagen type I antibody, followed by three successive washes in PBS in a humidified chamber. Cells were then blocked again in blocking buffer for 15 min followed by a 1-hour incubation in fluorescently conjugated secondary antibody. Cells were then washed again in PBS and the nuclei were counterstained with 4′,6-diamidino-2-phenylindole (DAPI). The cells were mounted onto microscope slides with Dako Cytomation fluorescent mounting medium (Dako). Digital fluorescent images were obtained using the Zeiss AxioVision SE64 Imaging software and analyzed using ImageJ2 software.

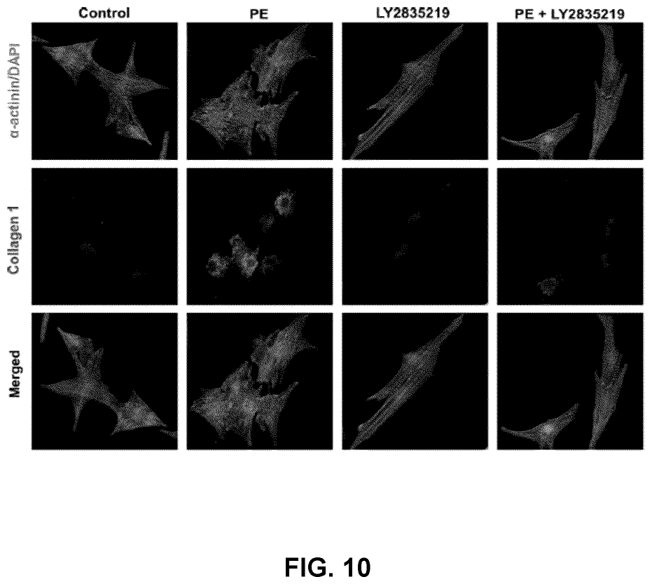

FIG. 10 shows that the PE treatment group resulted in increased expression of type I collagen compared to control. Treatment with drug LY2835219 leads to a decrease in expression of collagen type I. The effect of the LY2835219 was sustained in the combination treatment where drug LY2835219 following PE treatment resulted in decreased expression of collagen type I. As such, drug LY2835219 has a role in managing cardiac fibrosis which is a critical event in majority of cardiovascular complication including heart failure. The drug LY2835219 reduced the expression of collagen type I but was also able to override the pathological effect of PE as observed in the combination treatment as shown in FIG. 10.

### Example 10: LY2835219 and Cardiac Contractile Function

Calcium (Ca²⁺) signaling plays an essential role in several functions of cardiac myocytes. Rhythmic increases in intracellular Ca²⁺ concentration underlie the contractile function of the heart. Cardiac remodeling, along with altered Ca²⁺ handling, is seen in various cardiovascular complications and is often the first step in progression towards heart failure

Method: Freshly isolated wildtype neonatal rat ventricular cardiomyocytes were plated onto laminin coated glass coverslides, rinsed once and then loaded with 1 μM Fura2-AM (Life Technologies) for 15 min at 37 °C. After washing the cells thrice with Tyrode solution, measuring chambers were transferred onto a Nikon Eclipse TE2000-U microscope equipped with a fluorescence detection system (IonOptix). Ca²⁺ transient values were recorded according to the corresponding FRET experiments. F340/F380 values in Fura2-AM experiments were calculated from the ratios of 510 nm emission light measured at 340 and 380 nm excitations.

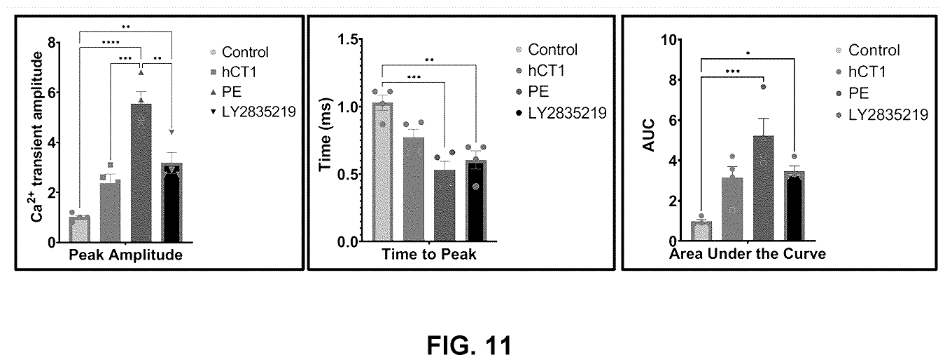

FIG. 11 shows that LY2835219 and hCT1 resulted in increase in calcium transient levels compared to control. PE treatment leads to aberrant increase calcium transient levels, suggestive of myocardial dysfunction. PE treatment resulted in decreased time to peak calcium transient, whereas hCT1 (positive control) and LY2835219 had similar time to peak levels. The area under the curve (AUC) represents the magnitude of response. hCT1 and LY2835219 had increased AUC compared to control, whereas PE treatment resulted in a much higher AUC, suggestive of defective contractile function of cardiomyocytes. The data suggests that hCT1 and LY28365219 improved contractility by modest increase in peak calcium transient levels and AUC with modest decrease in time to attain those peak levels. PE on the other hand lead to an abnormal increase of peak calcium transient levels which is typically a characteristic of defective contractile machinery.

### Example 11: LY2835219 and Cardiac Mitochondrial Function

Cardiac hypertrophy is commonly accompanied by severe oxidative damage, which is associated with energy depletion. The mitochondrion is the primary organelle for energy production and mitochondrial dysfunction contributes to the development of heart failure. In hypertrophied hearts, the activity of mitochondrial oxidative phosphorylation complexes leading to reduced oxygen consumption rate (OCR).

OCR detection of cardiomyocytes was conducted with the Seahorse Xfe 24 analyzer (Agilent, Santa Clara, USA). Cardiomyocytes were seeded at a density of 8×104/well in a special Seahorse 24-well plate (102342-100, Seahorse). After treating with hCT1, PE and drug for 24 h, the medium was replaced with XF medium containing 10 mM D-(+)-glucose (G7021, Sigma-Aldrich), and 1 mM sodium pyruvate (11360070, Thermo Fisher). The plates were placed in a CO₂-free 37 °C incubator for an hour before the measurement. A Seahorse XF Cell Mito Stress Test Kit (103015-100, Seahorse) was used for detection. The basal respiration of the NRVMs was detected, and ATP-linked OCR was analyzed after treatment with 1 μM oligomycin (ATP synthase inhibitor). Then, the uncoupling agent carbonyl cyanide 4-(trifluoromethoxy) phenylhydrazone (FCCP) 0.5 μM was injected to detect maximal respiration. Finally, rotenone and antimycin A (AR) were used to abolish mitochondrial respiration by inhibiting complexes I and III, respectively.

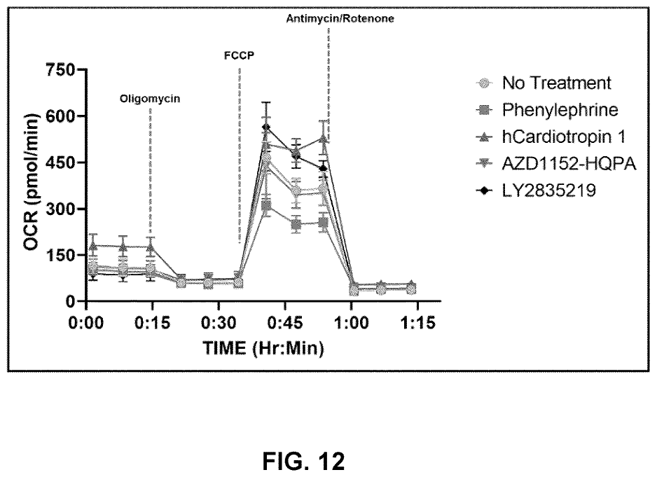

FIG. 12 shows that PE treatment resulted in a reduced oxygen consumption rate in cardiomyocyte. hCT1 (positive control) and LY2835219 were able to increase the rate of oxygen consumption, whereas drug AZD1152-HQPA had similar rate of oxygen consumption compared to untreated control. Since Oxygen Consumption Rate (OCR) is an established measure of mitochondrial function, the data suggest drug LY2835219 to improve oxygen consumption rate in cardiomyocytes which is suggestive of an enhanced mitochondrial function in cardiomyocytes.

### Example 12: LY2835219 and Cardiac Structural Remodeling

Animal Model Preparation. Sprague Dawley rats were continuously treated with phenylephrine (PE), an α-adrenergic agonist that causes vasoconstriction leading to increased afterload and decreased ventricular function, to create a left-ventricular dysfunction animal model. Phenylephrine causes myocardial hypertrophy of the LV, increased LV weight, systolic dysfunction, and increased expression of HF markers atrial natriuretic peptide and brain natriuretic peptide. For chronic infusion of phenylephrine, Alzet® mini-osmotic pumps (2002, Durect® Corp) containing Phenylephrine (PE) (10 mg/kg/day) or PBS vehicle were surgically implanted dorsally and subcutaneously in each Sprague Dawley rat under isoflurane anaesthesia. The animals were allowed to develop left ventricular hypertrophy for 2 weeks following the osmotic pump implantation.

Once the model was established (validated with echocardiography), the animals were treated with LY2835219 (75 mg/kg/day) daily via oral gavage for 3 weeks. Echocardiography was performed, the animals were sacrificed at 3 weeks endpoint and the blood and tissue were harvested for further analysis.

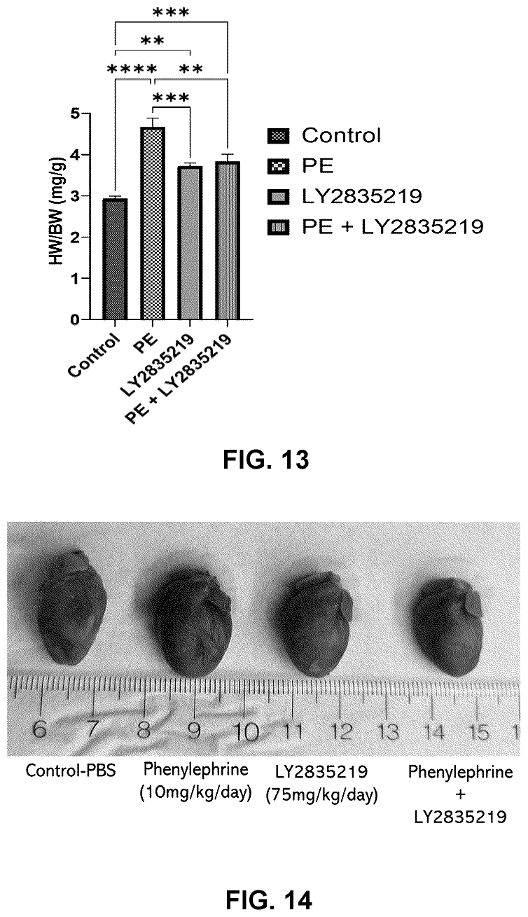

The ratio of Heart Weight/Body Weight was significantly increased in response to PE when compared to rats with sham (PBS), rats treated with LY2835219 alone, and rats with PE treatment for 2 weeks followed by 3 weeks of LY2835219 treatment as shown in FIG. 13. The same is evident from the whole heart image where the heart appears to be enlarged in PE treated group compared to the control and drug treatment group as shown in FIG. 14.

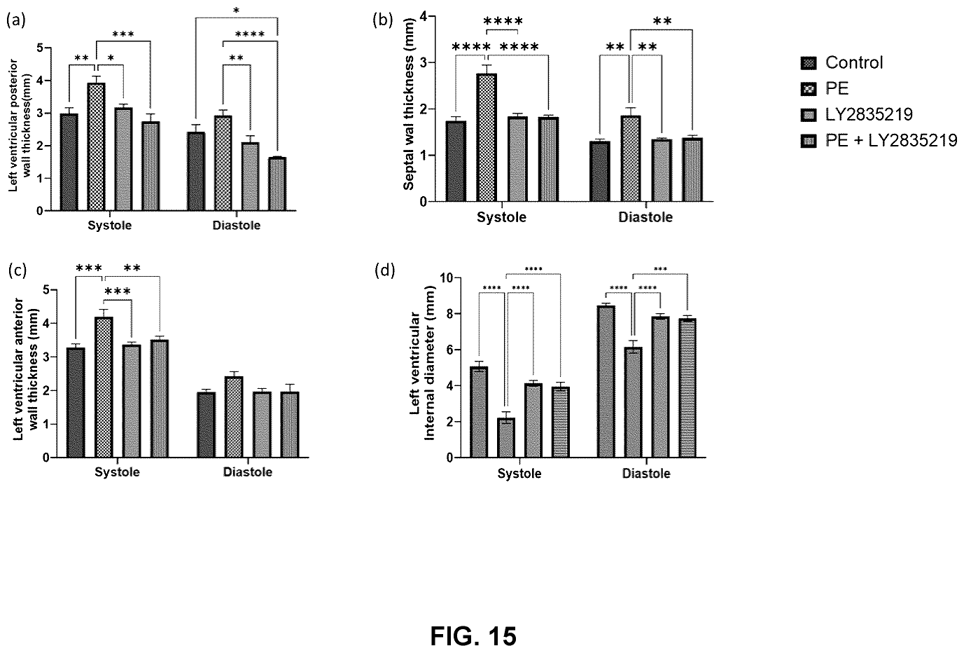

FIG. 15 shows the effect of LY2835219 on myocardial morphology using echocardiographic assessments via Vevo 2100 Imaging Platform. As shown in panels (a), (b), and (c), PE treatment led to increased thickness of left ventricular posterior wall, left ventricular anterior wall and septal wall at both systole and diastole compared to control animals and LY2835219 treatment group. This effect was reversed in PE treated rats subjected to 3 weeks of LY2835219 treatment. Furthermore, panel d similarly shows that the left ventricular internal diameter was reduced in PE treatment group whereas it was reversed with group subjected to PE for 2 weeks followed by LY2835219 treatment for 3 weeks.

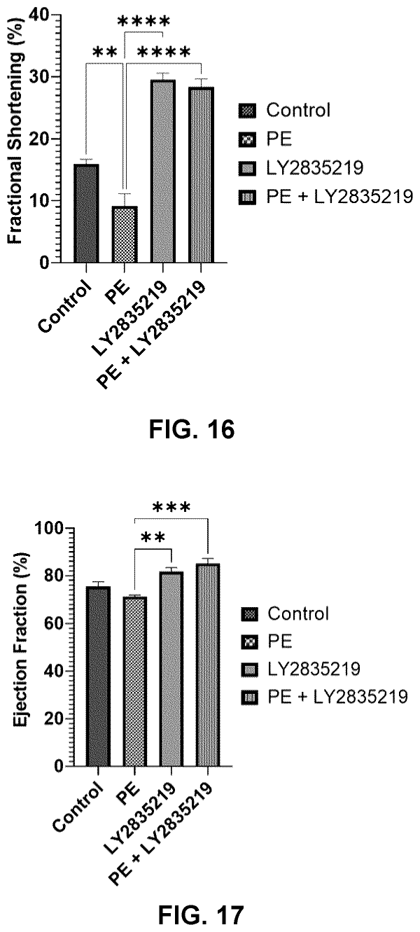

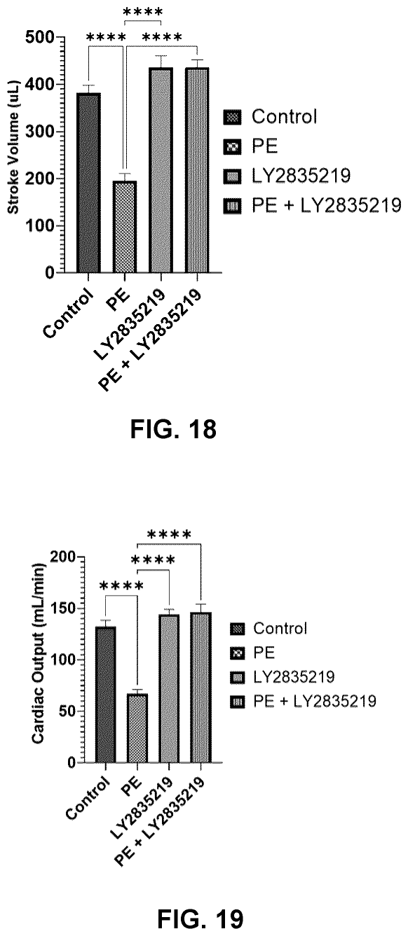

There are several key parameters that are determinants of cardiac function. Echocardiography revealed that PE treated rats had decreased ejection fraction (FIG. 16), percentage fractional shortening (FIG. 17), stroke volume (FIG. 18) and cardiac output (FIG. 19) after 2 weeks, whereas PE treated rats subjected to LY2835219 for 3 weeks had significantly enhanced cardiac function compared with control rats and PE treated rats. As such, LY2835219 has an anti-hypertrophic effect in phenylephrine induced left ventricular hypertrophy, and improved some of the key features of cardiac function such as ejection fraction, fractional shortening, stroke volume and cardiac output alone and in rats subjected to phenylephrine. The results suggest LY2835219 to be a potent agent in the management of cardiac hypertrophy and heart failure.

### Example 13—LY2835219 and Cardiac Fibrosis Remodelling

Cardiac fibrosis is a form of cardiac maladaptation characterized by the excessive accumulation of fibrous connective tissue in the heart which can lead to stiffness of the heart muscle and impaired heart function, potentially leading to heart failure. Phenylephrine has been reported to stimulate pathways involved in cardiac fibrosis, potentially leading to adverse effects on heart structure and function over time.

The rat model was prepared as described in Example 12, and the cardiac tissue harvested at the end of the 3 weeks was prepared with Masson's trichome staining methodology, which is a histological technique used to visualize and assess connective tissue, particularly collagen, within biological samples.

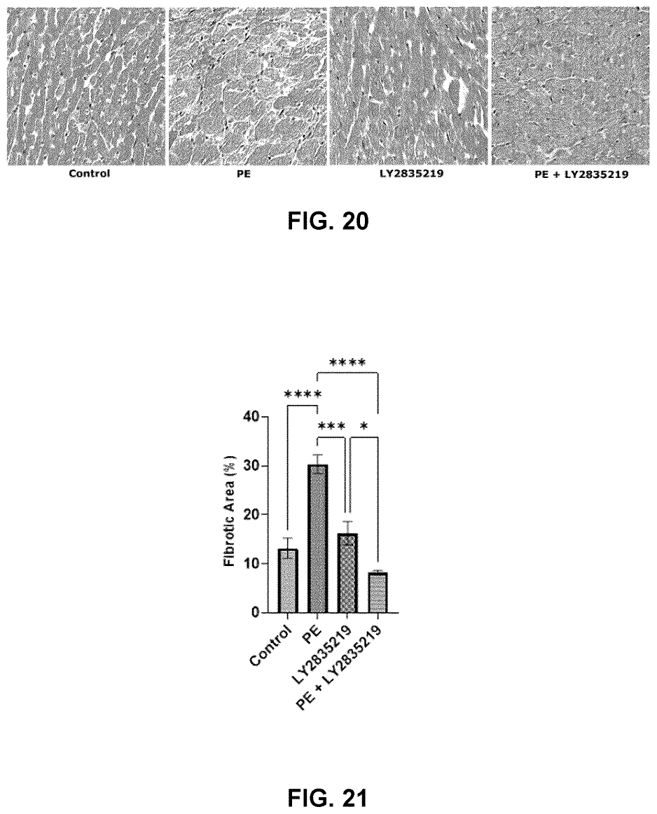

As shown in FIG. 20, PE led to a significant increase in cardiac fibrosis compared to control sham animals. LY2835219 treatment alone did not induce fibrosis, however LY2835219 treatment did reduce fibrosis in animals subjected to PE treatment, as also shown in FIG. 21, which is a quantification of the percentage fibrotic area in the histological images shown in FIG. 20. These results show the beneficial cardiac remodelling anti-fibrotic effect of LY2835219 in improving cardiac function under pathological cardiac remodeling.

### Example 14—LY2835219 and Cardiac Angiogenic Remodelling

Angiogenesis plays a vital role in supplying oxygen and nutrients to cardiac tissues, facilitating their proper function. In conditions such as ischemic heart disease or heart failure, where there's inadequate blood supply to the heart muscle, angiogenesis helps to improve blood flow, thereby enhancing cardiac function and potentially mitigating cardiac damage. CD31 staining is a common technique used to stain endothelial cells which drives angiogenesis by identifying and quantifying blood vessels.

The rat model was prepared as described in Example 12, and the cardiac tissue harvested at the end of the 3 weeks was prepared for CD31 Staining, which is a histological technique used to visualize and assess angiogenesis in cardiac tissue.

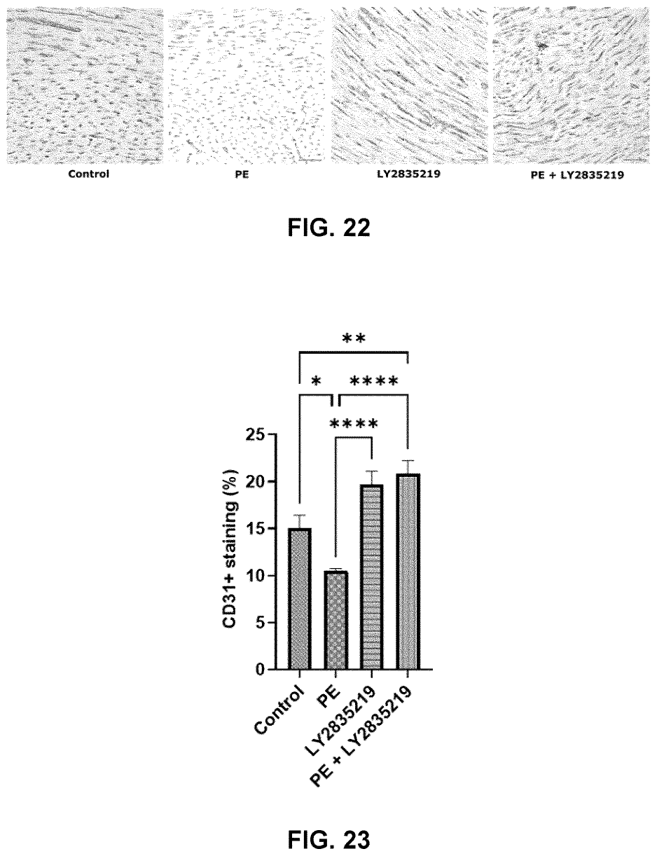

As shown in FIGS. 22 and 23, LY2835219 treatment leads to increased angiogenesis. PE treatments group observed significantly decreased CD31 staining suggesting compromised angiogenesis compared to the control sham animals. LY2835219 was also able to improve angiogenesis in PE treated animals suggesting its beneficial role in improving vasculature under pathological settings.

The above-described embodiments are intended to be examples only. Alterations, modifications and variations can be effected to the particular embodiments by those of skill in the art. The scope of the claims should not be limited by the particular embodiments set forth herein, but should be construed in a manner consistent with the specification as a whole.

In the preceding description, for purposes of explanation, numerous details are set forth in order to provide a thorough understanding of the embodiments. However, it will be apparent to one skilled in the art that these specific details are not required. In other instances, well-known electrical structures and circuits are shown in block diagram form in order not to obscure the understanding. For example, specific details are not provided as to whether the embodiments described herein are implemented as a software routine, hardware circuit, firmware, or a combination thereof.

### REFERENCES

Xia P, Liu Y, Chen J, Coates S, Liu D X, Cheng Z. Inhibition of cyclin-dependent kinase 2 protects against doxorubicin-induced cardiomyocyte apoptosis and cardiomyopathy. J Biol Chem. 2018; 293(51):19672-85.
Wang Z, Li J, Wang Y, Liu Q. Palbociclib improves cardiac dysfunction in diabetic cardiomyopathy by regulating Rb phosphorylation. Am J Transl Res. 2019; 11(6):3481-9.
Azzariti A, Bocci G, Porcelli L, Fioravanti A, Sini P, Simone G M, et al. Aurora B kinase inhibitor AZD1152: determinants of action and ability to enhance chemotherapeutics effectiveness in pancreatic and colon cancer. Br J Cancer. 2011; 104(5):769-80.
Lowenberg B, Muus P, Ossenkoppele G, Rousselot P, Cahn J Y, Ifrah N, et al. Phase 1/2 study to assess the safety, efficacy, and pharmacokinetics of Barasertib (AZD1152) in patients with advanced acute myeloid leukemia. Blood. 2011; 118(23):6030-6.
Gelbert L M, Cai S, Lin X, Sanchez-Martinez C, Del Prado M, Lallena M J, et al. Preclinical characterization of the CDK4/6 inhibitor LY2835219: in-vivo cell cycle-dependent/independent anti-tumor activities alone/in combination with gemcitabine. Invest New Drugs. 2014; 32(5):825-37.
Dorn G W, 2^{nd}. The fuzzy logic of physiological cardiac hypertrophy. Hypertension. 2007; 49(5):962-70.
Reid B G, Stratton M S, Bowers S, Cavasin M A, Demos-Davies K M, Susano I, et al. Discovery of novel small molecule inhibitors of cardiac hypertrophy using high throughput, high content imaging. J Mol Cell Cardiol. 2016; 97:106-13.
WO2020125513A1.
US2019298727A1.

All references cited herein are incorporated by references in their entireties.

1. (canceled)

2. A method of promoting cardiac remodeling in a subject, comprising:
administering to a subject a therapeutic agent comprising:
LY2835219 (Abemaciclib).

3. The method of claim 2, wherein the cardiac remodeling comprises left ventricular cardiac remodeling.

4. The method of claim 3, wherein the left ventricular cardiac remodeling comprises left ventricular eccentric hypertrophy.

5. The method of claim 2, wherein the cardiac remodeling comprises right ventricular cardiac remodeling.

6. The method of claim 5, wherein the right ventricular cardiac remodeling comprises right ventricular eccentric hypertrophy.

7. (canceled)

8. The method of claim 88, wherein the CDK4 inhibitor is
LY2835219 (Abemaciclib).

9. The method of claim 8, wherein the heart failure comprises left ventricular heart failure.

10. The method of claim 9, wherein the heart failure comprises left ventricular maladaptive hypertrophy.

11. The method of claim 9, wherein the left ventricular heart failure comprises left ventricular concentric hypertrophy.

12. The method of claim 8, wherein the heart failure comprises right ventricular heart failure.

13. (canceled)

14. The method of claim 12, wherein the right ventricular heart failure comprises right ventricular concentric hypertrophy.

15. The method of claim 88, wherein the heart failure is caused by myocardial infarction.

16. The method of claim 88, wherein the heart failure is caused by pulmonary arterial hypertension.

17-33. (canceled)

34. A use of LY2835219 (Abemaciclib) for preparation of a medicament for inducement of cardiac remodeling in a subject.

35. The use of claim 34, wherein the cardiac remodeling comprises left ventricular cardiac remodeling.

36. (canceled)

37. The use of claim 34, wherein the cardiac remodeling comprises right ventricular cardiac remodeling.

38-78. (canceled)

79. The method of claim 2, wherein the cardiac remodeling comprises reduction of cardiac fibrosis and/or improvement of cardiac angiogenesis.

80. The method of claim 88, wherein the heart failure is caused by cardiac fibrosis or impaired cardiac angiogenesis.

81. The method of claim 88, wherein the heart failure is caused by aortic stenosis.

82-87. (canceled)

88. A method of treating heart failure in a subject, comprising
administering to a subject a therapeutic agent comprising:
a CDK4 inhibitor.
